(19) United States
(12) Patent Application Publication
(10) Pub. No.: US 2024/0044887 A1
(43) Pub. Date: Feb. 8, 2024

(54) TARGET MATERIAL IDENTIFICATION METHOD

(71) Applicant: Taiwan Semiconductor Manufacturing Company, Ltd., Hsinchu (TW)

(72) Inventor: Tung-Tsun Chen, Hsinchu City (TW)

(73) Assignee: Taiwan Semiconductor Manufacturing Company, Ltd., Hsinchu (TW)

(21) Appl. No.: 17/880,676

(22) Filed: Aug. 4, 2022

(51) Int. Cl. G01N 33/543

(52) U.S. Cl. CPC G01N 33/54373

(57) ABSTRACT

A target material identification method includes the following steps. A bio-sensing integrated circuit having a sensor array is provided. The sensor array is divided into 1^{st}-N^{th} assays, and the 1^{st}-N^{th} assays are coated with different probes. A calibration process is performed to obtain 1^{st}-N^{th} pre-test measurement values respectively for the 1^{st}-N^{th} assays. A sample fluid having the target material therein is provided onto the 1^{st}-N^{th} assays. A bio-sensing process is performed on the sample fluid to obtain 1^{st}-N^{th} post-test measurement values respectively for the 1^{st}-N^{th} assays. The 1^{st}-N^{th} pre-test measurement values are compared with the corresponding 1^{st}-N^{th} post-test measurement values, so as to determine whether the target material is bind to the probes in each of the 1^{st}-N^{th} assays. An assay having the target material bind to the probe is marked as a binding assay. The target material is identified based on the probe in the binding assay.

### BACKGROUND

Biosensors are devices for sensing and detecting biomolecules and operate on the basis of electronic, electrochemical, optical, and mechanical detection principles. Biosensors that include transistors are sensors that electrically sense charges, photons, and mechanical properties of bio-entities or biomolecules. The detection can be performed by detecting the bio-entities or biomolecules themselves, or through interaction and reaction between specified reactants and bio-entities/biomolecules. Such biosensors can be manufactured using semiconductor processes, can quickly convert electric signals, and can be easily applied to integrated circuits (ICs).

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a schematic cross-sectional view of a bio-sensing integrated circuit in accordance with some embodiments of the disclosure.

FIG. 2 is a schematic partial enlarged view of the bio-sensing integrated circuit in FIG. 1 during a bio-sensing process.

FIG. 3A to FIG. 3C are schematic views of various cross-linkers attaching to the sensing layer of the bio-sensing integrated circuit in FIG. 1.

FIG. 4 is an example diagram of a sensor array and a schematic circuit diagram of the sensor array.

FIG. 5A to FIG. 5F are schematic top views illustrating various stages of a coating process for coating probes onto the bio-sensing integrated circuit in FIG. 1.

FIG. 6 is a schematic flow of a target material identification method in accordance with some embodiments of the disclosure.

FIG. 7 is a current vs. time curve of the sample fluid in the target material identification method of FIG. 6.

FIG. 8 is a schematic flow of a target material identification method in accordance with some alternative embodiments of the disclosure.

FIG. 9A is a current vs. time curve of the sample fluid in the first assay in the target material identification method of FIG. 8.

FIG. 9B is a current vs. time curve of the sample fluid in the second assay in the target material identification method of FIG. 8.

FIG. 9C is a current vs. time curve of the sample fluid in the third assay in the target material identification method of FIG. 8.

FIG. 9D is a current vs. time curve of the sample fluid in the fourth assay in the target material identification method of FIG. 8.

FIG. 10 is a schematic flow of a target material identification method in accordance with some alternative embodiments of the disclosure.

FIG. 11 is a current vs. time curve of the sample fluid in the target material identification method of FIG. 10.

FIG. 12 is a schematic flow of a target material identification method in accordance with some alternative embodiments of the disclosure.

FIG. 13A is a current vs. time curve of the sample fluid in the first sensor array in the target material identification method of FIG. 12.

FIG. 13B is a current vs. time curve of the sample fluid in the second sensor array in the target material identification method of FIG. 12.

FIG. 13C is a current vs. time curve of the sample fluid in the third sensor array in the target material identification method of FIG. 12.

FIG. 13D is a current vs. time curve of the sample fluid in the fourth sensor array in the target material identification method of FIG. 12.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

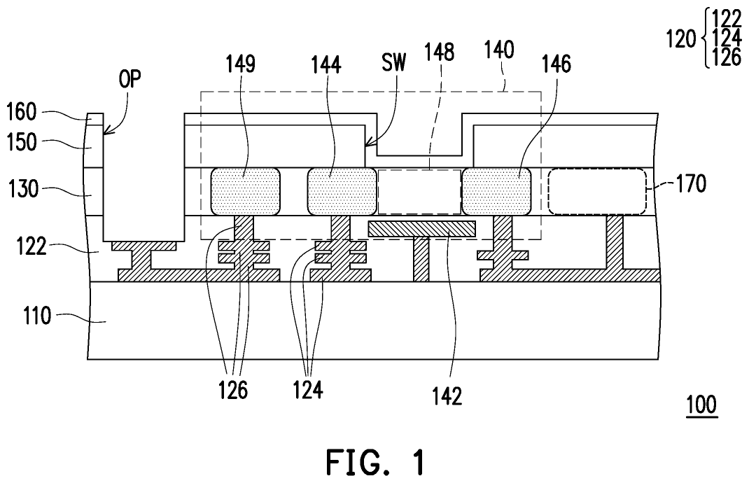

FIG. 1 is a schematic cross-sectional view of a bio-sensing integrated circuit 100 in accordance with some embodiments of the disclosure. Referring to FIG. 1, the bio-sensing integrated circuit 100 includes a carrier substrate 110, an interconnect structure 120, a semiconductor substrate 130, a Biosensor Field-Effect Transistor (BioFET) 140, a passivation layer 150, a sensing layer 160, and a circuitry 170.

In some embodiments, the carrier substrate 110 is a bulk semiconductor substrate, such as a bulk substrate of monocrystalline silicon. As illustrated in FIG. 1, the interconnect structure 120 is disposed on the carrier substrate 110. In some embodiments, the interconnect structure 120 includes a dielectric layer 122, a plurality of conductive patterns 124, and a plurality of conductive vias 126. In some embodiments, a material of the dielectric layer 122 includes polyimide, epoxy resin, acrylic resin, phenol resin, benzocyclobutene (BCB), polybenzooxazole (PBO), or any other suitable polymer-based dielectric material. Alternatively, the dielectric layer 122 may be formed of oxides or nitrides, such as silicon oxide, silicon nitride, aluminum oxide, hafnium oxide, hafnium zirconium oxide, or the like. The dielectric layer 122 may be formed by suitable fabrication techniques, such as spin-on coating, chemical vapor deposition (CVD), plasma-enhanced chemical vapor deposition (PECVD), or the like. For simplicity, the dielectric layer 122 is illustrated as a bulky layer in FIG. 1, but it should be understood that the dielectric layer 122 may be constituted by multiple dielectric layers. In some embodiments, the conductive patterns 124 and the conductive vias 126 are embedded in the dielectric layer 122. In some embodiments, the conductive patterns 124 located at different level heights are connected to one another through the conductive vias 126. In other words, the conductive patterns 124 are electrically connected to one another through the conductive vias 126. In some embodiments, a material of the conductive patterns 124 and the conductive vias 126 includes aluminum, titanium, copper, nickel, tungsten, or alloys thereof. The conductive patterns 124 and the conductive vias 126 may be formed by electroplating, deposition, and/or photolithography and etching. In some embodiments, the conductive patterns 124 and the conductive vias 126 are formed simultaneously. It should be noted that the number of the conductive patterns 124 and the number of the conductive vias 126 illustrated in FIG. 1 are merely for illustrative purposes, and the disclosure is not limited thereto. In some alternative embodiments, fewer or more layers of the conductive patterns 124 and/or the conductive vias 126 may be formed depending on the circuit design. In some embodiments, the interconnect structure 120 is referred to as “back-end-of-line (BEOL) interconnect structure.”

In some embodiments, the semiconductor substrate 130 is disposed on the interconnect structure 120. The semiconductor substrate 130 accommodates the BioFET 140 and may be, for example, a semiconductor layer of a semiconductor-on-insulator (SOI) substrate or a bulk semiconductor substrate.

As illustrated in FIG. 1, the BioFET 140 includes a gate electrode 142, a source region 144, a drain region 146, a channel region 148, and a body region 149. In some embodiments, the gate electrode 142 is embedded in the interconnect structure 120. Moreover, the gate electrode 142 is electrically connected to the interconnect structure 120. For example, the gate electrode 142 is in physical contact with some of the conductive vias 126 such that the gate electrode 142 is electrically connected to the conductive patterns 124 and the conductive vias 126 of the interconnect structure 120. In some embodiments, a material of the gate electrode 142 includes polysilicon, metal, metal alloy, or a combination thereof. As illustrated in FIG. 1, the source region 144 and the drain region 146 are embedded in the semiconductor substrate 130. The source region 144 and the drain region 146 may be respectively doped with p-type dopants, such as boron or BF₂; n-type dopants, such as phosphorus or arsenic; and/or a combination thereof. In some embodiments, the source region 144 and the drain region 146 are electrically connected to the interconnect structure 120. For example, the source region 144 and the drain region 146 are in physical contact with some of the conductive vias 126 such that the source region 144 and the drain region 146 are electrically connected to the conductive patterns 124 and the conductive vias 126 of the interconnect structure 120. In some embodiments, the channel region 148 is also embedded in the semiconductor substrate 130. For example, the source region 144 and the drain region 146 may respectively locate on two opposite sides of the channel region 148. In some embodiments, the channel region 148 is a doped region. For example, the channel region 148 may be doped with p-type dopants, such as boron or BF₂; n-type dopants, such as phosphorus or arsenic; and/or a combination thereof. In some embodiments, the doping type of the channel region 148 is different from the doping type of the source region 144 and the drain region 146. In some embodiments, the source region 144, the drain region 146, and the channel region 148 extend continuously from a top surface of the semiconductor substrate 130 to a bottom surface of the semiconductor substrate 130. On the other hand, the gate electrode 142 is arranged under the semiconductor substrate 130. In some embodiments, the gate electrode 142 is arranged laterally between the source region 144 and the drain region 146, and is spaced apart from the semiconductor substrate 130 by a gate dielectric layer (for example, part of the dielectric layer 122).

In some embodiments, the body region 149 is adjacent to the source region 144. For example, the body region 149 is embedded in the semiconductor substrate 130. In some embodiments, the body region 149 is electrically connected to the interconnect structure 120. For example, the body region 149 is in physical contact with some of the conductive vias 126 such that the body region 149 is electrically connected to the conductive patterns 124 and the conductive vias 126 of the interconnect structure 120. In some embodiments, the body region 149 is used to bias the carrier concentration in the channel region 148. As such, a negative voltage bias may be applied to the body region 149 to improve the sensitivity of the BioFET 140. In some embodiments, the body region 149 is electrically grounded. However, the disclosure is not limited thereto. In some alternative embodiments, the body region 149 is electrically connected to the source region 144.

As illustrated in FIG. 1, the passivation layer 150 is disposed over the semiconductor substrate 130. In some embodiments, the passivation layer 150 includes a sensing well SW. The sensing well SW extends into the passivation layer 150 to proximate the channel region 148. For example, the sensing well SW extends through the passivation layer 150 to expose the channel region 148. In some embodiments, the passivation layer 150 includes silicon dioxide, a buried oxide (BOX) layer of a SOI substrate, some other dielectrics, or a combination thereof.

In some embodiments, the sensing layer 160 is disposed on the passivation layer 150. For example, the sensing layer 160 covers the passivation layer 150 and extends into the sensing well SW to be in physical contact with the channel region 148. In some embodiments, the sensing layer 160 is configured to react with or bind to bio-entities to facilitate a change in the conductance of the channel region 148, such that the presence of the bio-entities may be detected based on the conductance of the channel region 148. In some embodiments, a material of the sensing layer 160 includes hafnium oxide, titanium nitride, titanium, a high-k dielectric, some other materials configured to react with or bind to the bio-entities, or a combination thereof. In some embodiments, the high-k dielectric is a dielectric with a dielectric constant that is greater than about 3.9. The bio-entities may be, for example, DNA, ribonucleic acid (RNA), drug molecules, enzymes, proteins, antibodies, antigens, or a combination thereof. In some embodiments, the sensing layer 160 has a thickness of less than about 100 nm.

In some embodiments, the circuitry 170 is embedded in the semiconductor substrate 130 and is adjacent to the drain region 146. In some embodiments, the circuitry 170 is separated from the drain region 146. In some embodiments, the circuitry 170 includes any number of Metal-Oxide-Semiconductor Field-Effect Transistor (MOSFET) devices, resistors, capacitors, or inductors to form circuitry to aid in the operation of the bio-sensing integrated circuit 100. In some embodiments, the circuitry 170 may be optional.

As illustrated in FIG. 1, the bio-sensing integrated circuit 100 further includes a pad opening OP. In some embodiments, the pad opening OP penetrates through the sensing layer 160, the passivation layer 150, and the semiconductor substrate 130. In some embodiments, the pad opening OP further extend into a portion of the interconnect structure 120. For example, the pad opening OP penetrate through a portion of the dielectric layer 122 to expose one of the topmost conductive patterns 124.

For simplicity, one BioFET 140 and one sensing well SW is shown in FIG. 1. However, it should be understood that multiple BioFETs 140 and multiple sensing wells SW may be found in the bio-sensing integrated circuit 100. When multiple BioFETs 140 and multiple sensing wells SW are presented in the bio-sensing integrated circuit 100, the sensing wells SW may be arranged to match the corresponding BioFET 140. For example, each sensing well SW may correspond to one BioFET 140. However, the disclosure is not limited thereto. In some alternative embodiments, each sensing well SW may have multiple BioFETs 140 directly underneath it. In some embodiments, a plurality of shallow trench isolation (STI) regions (not shown) may be embedded in the semiconductor substrate 130 to isolate two adjacent BioFETs 140.

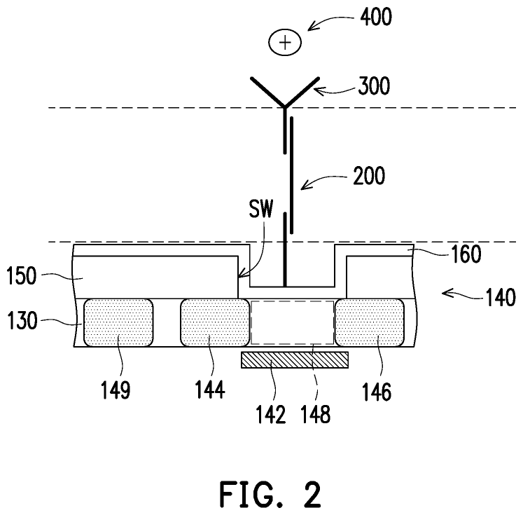

FIG. 2 schematic partial enlarged view of the bio-sensing integrated circuit 100 in FIG. 1 during a bio-sensing process. Referring to FIG. 2, a cross-linker 200 is provided on the sensing layer 160. In some embodiments, a probe 300 is attached to the cross-linker 200 for capturing a target material 400. Please be noted that the drawings shown in FIG. 2 is not to scale, and in some embodiments, the cross-linker 200 and the probe 300 are provided within the sensing well SW. Referring to FIG. 1 and FIG. 2, during the bio-sensing process, a sample fluid (not shown) is provided on the bio-sensing integrated circuit 100. For example, the sample fluid flows into the sensing well SW such that the target material 400 in the sample fluid is bind to the probe 300. Due to the binding between the target material 400 and the probe 300, the conductance of the channel region 148 underneath the sensing well SW would change. As such, the presence of the target material 400 may be detected based on the conductance of the channel region 148.

In some embodiments, the probe 300 includes Protein-based probe, DNA-based probed, Peptide-based probe, and/or Aptamer-based probe. The Protein-based probe includes Recombinant proteins. The DNA-based probe includes Complementary DNAs. The Peptide-based probe includes Synthetized peptides. The Aptamer-based probe includes Synthetized aptamers. Examples of the probe 300 includes Spike RBD probe protein, Nucleocapsid probe protein, SARS-Cov-1 probe protein, MERS probe protein, Influenza A probe protein, Influenza B probe protein, Influenza C probe protein, Influenza D probe protein, Cytokine protein, Phosphorylate Tau (P-Tau)-181, Phosphorylated Tau (P-Tau)-217, T-Tau, Beta-amyloid protein (Aβ), Aβ-40, Aβ-42, Alpha-Synuclein (α-Synuclein), Oligomeric α-syn, and TDP-43.

In some embodiments, the target material 400 includes SARS-Cov-2 antigen, SARS-Cov-1 antigen, MERS-Cov-1 antigen, Influenza A antigen, Influenza B antigen, Influenza C antigen, Influenza D antigen, Cytokine storm, Alzheimer's Disease antibody, Parkinson's Disease antibody, and Frontotemporal Dementia (FTD). In some embodiments, each of the target materials 400 correspond to a specific probe 300 listed above. That is, each target material 400 would only bind to a specific probes 300 list above. For example, the SARS-Cov-2 antigen corresponds to the Spike RBD probe protein and the Nucleocapsid probe protein. The SARS-Cov-1 antigen corresponds to the SARS-Cov-1 probe protein. The MERS-Cov-1 antigen corresponds to the MERS probe protein. The Influenza A antigen corresponds to the Influenza A probe protein. The Influenza B antigen corresponds to the Influenza B probe protein. The Influenza C antigen corresponds to the Influenza C probe protein. The Influenza D antigen corresponds to the Influenza D probe protein. The Cytokine storm corresponds to the Cytokine protein. The Alzheimer's Disease antibody corresponds to the Phosphorylate Tau (P-Tau)-181, the Phosphorylated Tau (P-Tau)-217, the T-Tau, the Beta-amyloid protein (Aβ), the Aβ-40, and the Aβ-42. The Parkinson's Disease antibody corresponds to the Alpha-Synuclein (α-Synuclein) and the Oligomeric α-syn. The FTD corresponds to the TDP-43.

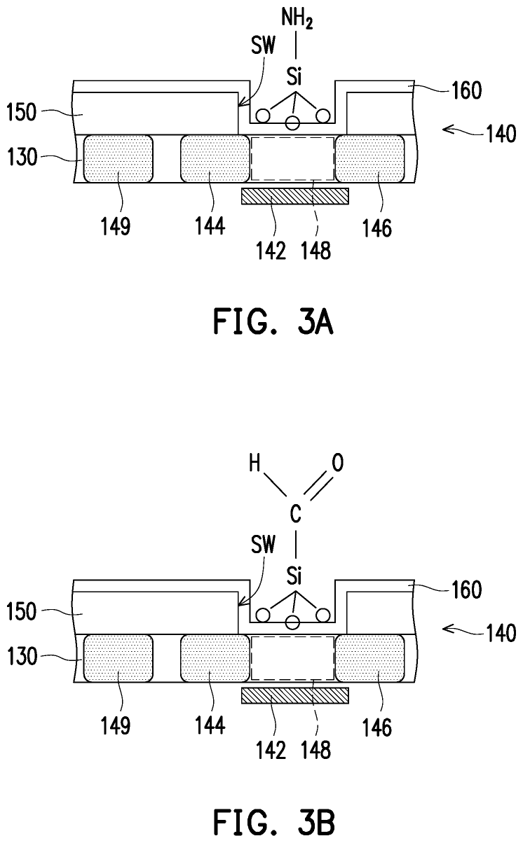

In some embodiments, the cross-linker 200 includes a combination of an amino group and a silane group, a combination of an aldehyde group and a silane group, or a combination of a thiol group and a silane group. The molecular structures of various cross-linkers 200 are shown in FIG. 3A to FIG. 3C. FIG. 3A to FIG. 3C are schematic views of various cross-linkers 200 attaching to the sensing layer 160 of the bio-sensing integrated circuit 100 in FIG. 1. Referring to FIG. 3A, amino silanization in which an amino group binding to a silane group is shown. Referring to FIG. 3B, aldehyde silanization in which an aldehyde group binding to a silane group is shown. Referring to FIG. 3C, thiol silanization in which a thiol group binding to a silane group is shown.

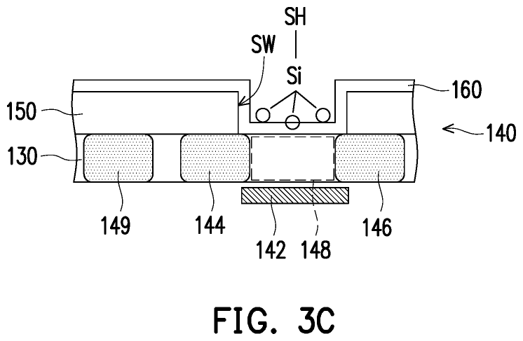

FIG. 4 is an example diagram of a sensor array SA and a schematic circuit diagram of the sensor array SA. In some embodiments, the sensor array SA is provided by the bio-sensing integrated circuit 100 discussed above. For example, when multiple BioFETs 140 and multiple sensing wells SW are presented in the bio-sensing integrated circuit 100, the sensing wells SW and the BioFETs 140 may be arranged in an array to form the sensor array SA of FIG. 4. As illustrated in FIG. 4, the sensor array SA includes, for example, 10 columns and 10 rows. In some embodiments, each row includes 10 pixels PX. Meanwhile, each column also includes 10 pixels PX. In some embodiments, each pixel PX corresponds to one sensing well SW and one bioFET 140 shown in FIG. 1. In some embodiments, each pixel PX in the sensor array SA corresponds to a particular column and a particular row. In some embodiments, a column decoder CD provides a column selection signal to the pixels PX in the sensor array SA, and a row decoder RD provides a row selection signal to the pixels PX in the sensor array SA. For example, a selective switch SWT corresponding to a particular pixel PX is electrically connected to both the column decoder CD and the row decoder RD. The selective switch SWT may be turned on in response to a selection signal provided by the column decoder CD and the row decoder RD, and in turn enables the bio-sensing process of the corresponding pixel PX. As illustrated in FIG. 4, the sensor array SA is electrically coupled to a Trans-impedance Amplifier (TIA) 500. In some embodiments, the measurement values (for example, a current between the source region 144 and the drain region 146) obtained from each pixel PX during the bio-sensing process are sequentially transmitted to the TIA 500. Subsequently, the TIA 500 enhances and magnifies the signal quality to improve the detection ability of the sensor array SA.

In some embodiments, the bio-sensing process described above may be adopted in a target material identification method. For example, as mentioned above, a specific type of target material 400 would only bind to a certain type of probe 300. Moreover, when the target material 400 is bind to the probe 300 during the bio-sensing process, the conductance of the channel region 148 underneath the sensing well SW would change, and the binding of the target material 400 may be detected based on the conductance of the channel region 148. As such, by controlling the type of probe 300 and by observing the change in the conductance of the channel region 148, the type of the target material 400 in a sample fluid may be determined. In some embodiments, the target material identification method includes the following steps. A sample fluid having a target material 400 therein is provided. In addition, at least one bio-sensing integrated circuit 100 as shown in FIGS. 1 and 4 is provided as well. The at least one bio-sensing integrated circuit 100 may provide a 1^{st} assay to an N^{th} assay. Then, a coating process is performed on the at least one bio-sensing integrated circuit 100 to coat the pixels PX (i.e. the sensing wells SW) in the 1^{st} assay to the N^{th} assay respectively with different probes 300. A calibration process is performed on the 1^{st} assay to the N^{th} assay to obtain a 1^{st} pre-test measurement value to an N^{th} pre-test measurement value respectively for the 1^{st} assay to the N^{th} assay. In some embodiments, the 1^{st} pre-test measurement value to the N^{th} pre-test measurement value are currents between the source region 144 and the drain region 146 respectively in the 1^{st} assay to the N^{th} assay. Then, the sample fluid is applied to the 1^{st} assay to the N^{th} assay. Subsequently, a bio-sensing process is performed on the sample fluid by the at least one bio-sensing integrated circuit 100 to obtain a 1^{st} post-test measurement value to an N^{th} post-test measurement value respectively for the 1^{st} assay to the N^{th} assay. In some embodiments, the 1^{st} post-test measurement value to the N^{th} post-test measurement value are currents between the source region 144 and the drain region 146 respectively in the 1^{st} assay to the N^{th} assay after the sample fluid is applied. Then, the 1^{st} pre-test measurement value to the N^{th} pre-test measurement value are respectively compared with the corresponding 1^{st} post-test measurement value to the corresponding N^{th} post-test measurement value to determine whether the target material 400 is bind to the probe 300 in each of the 1^{st} assay to the N^{th} assay. Thereafter, an assay among the 1^{st} assay to the N^{th} assay having the target material 400 bind to the probe 300 is marked as a binding assay. Subsequently, the target material 400 may be identified based on the probe 300 in the binding assay.

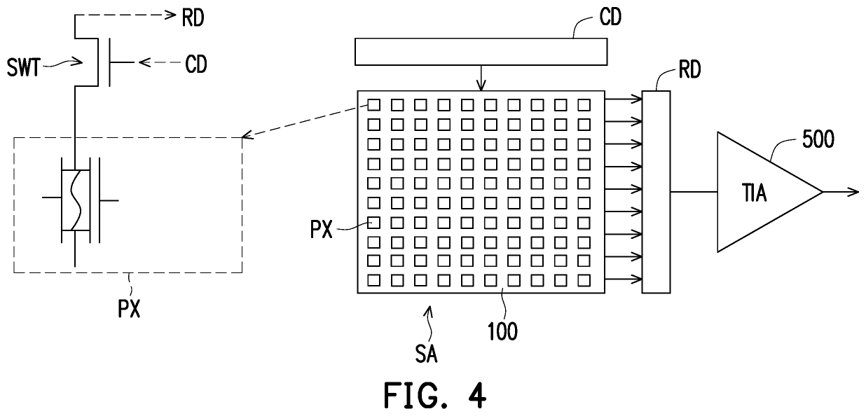

As mentioned above, the pixels PX (i.e. the sensing wells SW) in the 1^{st} assay to the N^{th} assay are respectively coated with different probes 300. The method of coating different probes 300 in different assays will be exemplified in detail below in conjunction with FIG. 5A to FIG. 5E.

FIG. 5A to FIG. 5F are schematic top views illustrating various stages of a coating process for coating probes 300a, 300b, 300c, and 300c onto the bio-sensing integrated circuit 100 in FIG. 1.

Referring to FIG. 5A, the bio-sensing integrated circuit 100 discussed above is provided. In some embodiments, the sensor array SA of the bio-sensing integrated circuit 100 is being divided into four regions. The first region corresponds to a first assay A1, the second region corresponds to a second assay A2, the third region corresponds to a third assay A3, and the fourth region corresponds to a fourth assay A4. In some embodiments, the first assay A1, the second assay A2, the third assay A3, and the fourth assay A4 respectively include multiple pixels PX to ensure the detection precision.

In some embodiments, a surface activation process is performed on the sensor array SA of the bio-sensing integrated circuit 100. In some embodiments, the surface activation process is performed in a gas-phase. For example, the bio-sensing integrated circuit 100 may be placed in a chamber (not shown), and a surface activation gas may be passed into the chamber to activate the sensing layer 160 in each of the pixels PX (i.e. the sensing wells SW) of the sensor array SA. In some embodiments, the surface activation process is performed globally on the sensor array SA. For example, the pixels PX in the first assay A1, the second assay A2, the third assay A3, and the fourth assay A4 are all subjected to the same surface activation gas at once. In some embodiments, the activation gas includes O₂ gas, silane gas, O₃ gas, or the like. In some embodiments, the surface activation process enhances the bonding strength between the sensing layer 160 and the subsequently coated probes.

Referring to FIG. 5B, a first mask layer MSK1 is placed on the sensor array SA of the bio-sensing integrated circuit 100. In some embodiments, the first mask layer MSK1 has a plurality of openings OP1. The locations of the openings OP1 correspond to the pixels PX in the first assay A1. For example, the openings OP1 of the first mask layer MSK expose the pixels PX located in the first assay A1. Meanwhile, the first mask layer MSK1 completely covers the second assay A2, the third assay A3, and the fourth assay A4. In other words, the pixels PX in the second assay A2, the third assay A3, and the fourth assay A4 are being shielded/covered by the first mask layer MSK1.

After the sensor array SA is covered by the first mask layer MSK1, a first probe solution is applied to the sensor array SA and the first mask layer MSK1. In some embodiments, the first probe solution includes a first solvent and a first probe 300a dissolved in the first solvent. In some embodiments, the first solvent includes phosphate-buffered saline (BPS) or the like. On the other hand, the first probe 300a includes any one of the probes 300 listed above. Since the openings OP1 of the first mask layer MSK1 expose the pixels PX in the first assay A1, the first probe solution is coated onto the sensing layer 160 of the pixels PX (i.e. the sensing wells SW) in the first assay A1. On the other hand, since the pixels PX in the second assay A2, the third assay A3, and the fourth assay A4 are being protected by the first mask layer MSK1, the first probe solution is not coated onto the pixels PX in the second assay A2, the third assay A3, and the fourth assay A4. In some embodiments, since the sensing layer 160 is being activated in the step shown in FIG. 5A, the first probe 300a in the first probe solution is bonded to the sensing layer 160 of the pixels PX in the first assay A1. Thereafter, a suction process is performed on the first probe solution to remove the first solvent, thereby allowing the first probe 300a to be coated onto the pixels PX in the first assay A1. It should be noted that since the first probe 300a is bonded to the sensing layer 160 of the pixels PX in the first assay A1, the suction process would only remove the first solvent and would not remove the first probe 300a. After the first probe 300a is coated onto the pixels PX in the first assay A1, the first mask layer MSK1 is removed.

Referring to FIG. 5C, a second mask layer MSK2 is placed on the sensor array SA of the bio-sensing integrated circuit 100. In some embodiments, the second mask layer MSK2 has a plurality of openings OP2. The locations of the openings OP2 correspond to the pixels PX in the second assay A2. For example, the openings OP2 of the second mask layer MSK2 expose the pixels PX located in the second assay A2. Meanwhile, the second mask layer MSK2 completely covers the first assay A1, the third assay A3, and the fourth assay A4. In other words, the pixels PX in the first assay A1, the third assay A3, and the fourth assay A4 are being shielded/covered by the second mask layer MSK2.

After the sensor array SA is covered by the second mask layer MSK2, a second probe solution is applied to the sensor array SA and the second mask layer MSK2. In some embodiments, the second probe solution includes a second solvent and a second probe 300b dissolved in the second solvent. In some embodiments, the second solvent includes BPS or the like. On the other hand, the second probe 300b is different from the first probe 300a. For example, the second probe 300b includes any one of the probes 300 listed above, as long as the second probe 300b is different from the first probe 300a. Since the openings OP2 of the second mask layer MSK2 expose the pixels PX in the second assay A2, the second probe solution is coated onto the sensing layer 160 of the pixels PX (i.e. the sensing wells SW) in the second assay A2. On the other hand, since the pixels PX in the first assay A1, the third assay A3, and the fourth assay A4 are being protected by the second mask layer MSK2, the second probe solution is not coated onto the pixels PX in the first assay A1, the third assay A3, and the fourth assay A4. In some embodiments, since the sensing layer 160 is being activated in the step shown in FIG. 5A, the second probe 300b in the second probe solution is bonded to the sensing layer 160 of the pixels PX in the second assay A2. Thereafter, a suction process is performed on the second probe solution to remove the second solvent, thereby allowing the second probe 300b to be coated onto the pixels PX in the second assay A2. It should be noted that since the second probe 300b is bonded to the sensing layer 160 of the pixels PX in the second assay A2, the suction process would only remove the second solvent and would not remove the second probe 300b. After the second probe 300b is coated onto the pixels PX in the second assay A2, the second mask layer MSK2 is removed.

Referring to FIG. 5D, a third mask layer MSK3 is placed on the sensor array SA of the bio-sensing integrated circuit 100. In some embodiments, the third mask layer MSK3 has a plurality of openings OP3. The locations of the openings OP3 correspond to the pixels PX in the third assay A3. For example, the openings OP3 of the third mask layer MSK3 expose the pixels PX located in the third assay A3. Meanwhile, the third mask layer MSK3 completely covers the first assay A1, the second assay A2, and the fourth assay A4. In other words, the pixels PX in the first assay A1, the second assay A2, and the fourth assay A4 are being shielded/covered by the third mask layer MSK3.

After the sensor array SA is covered by the third mask layer MSK3, a third probe solution is applied to the sensor array SA and the third mask layer MSK3. In some embodiments, the third probe solution includes a third solvent and a third probe 300c dissolved in the third solvent. In some embodiments, the third solvent includes BPS or the like. On the other hand, the third probe 300c is different from the first probe 300a and the second probe 300b. For example, the third probe 300c includes any one of the probes 300 listed above, as long as the third probe 300c is different from the first probe 300a and the second probe 300b. Since the openings OP3 of the third mask layer MSK3 expose the pixels PX in the third assay A3, the third probe solution is coated onto the sensing layer 160 of the pixels PX (i.e. the sensing wells SW) in the third assay A3. On the other hand, since the pixels PX in the first assay A1, the second assay A2, and the fourth assay A4 are being protected by the third mask layer MSK3, the third probe solution is not coated onto the pixels PX in the first assay A1, the second assay A2, and the fourth assay A4. In some embodiments, since the sensing layer 160 is being activated in the step shown in FIG. 5A, the third probe 300c in the third probe solution is bonded to the sensing layer 160 of the pixels PX in the third assay A3. Thereafter, a suction process is performed on the third probe solution to remove the third solvent, thereby allowing the third probe 300c to be coated onto the pixels PX in the third assay A3. It should be noted that since the third probe 300c is bonded to the sensing layer 160 of the pixels PX in the third assay A3, the suction process would only remove the third solvent and would not remove the third probe 300c. After the third probe 300c is coated onto the pixels PX in the third assay A3, the third mask layer MSK3 is removed.

Referring to FIG. 5E, a fourth mask layer MSK4 is placed on the sensor array SA of the bio-sensing integrated circuit 100. In some embodiments, the fourth mask layer MSK4 has a plurality of openings OP4. The locations of the openings OP4 correspond to the pixels PX in the fourth assay A4. For example, the openings OP4 of the fourth mask layer MSK4 expose the pixels PX located in the fourth assay A4. Meanwhile, the fourth mask layer MSK4 completely covers the first assay A1, the second assay A2, and the third assay A3. In other words, the pixels PX in the first assay A1, the second assay A2, and the third assay A3 are being shielded/covered by the fourth mask layer MSK4.

After the sensor array SA is covered by the fourth mask layer MSK4, a fourth probe solution is applied to the sensor array SA and the fourth mask layer MSK4. In some embodiments, the fourth probe solution includes a fourth solvent and a fourth probe 300d dissolved in the fourth solvent. In some embodiments, the fourth solvent includes BPS or the like. On the other hand, the fourth probe 300d is different from the first probe 300a, the second probe 300b, and the third probe 300c. For example, the fourth probe 300d includes any one of the probes 300 listed above, as long as the fourth probe 300d is different from the first probe 300a, the second probe 300b, and the third probe 300c. Since the openings OP4 of the fourth mask layer MSK4 expose the pixels PX in the fourth assay A4, the fourth probe solution is coated onto the sensing layer 160 of the pixels PX (i.e. the sensing wells SW) in the fourth assay A4. On the other hand, since the pixels PX in the first assay A1, the second assay A2, and the third assay A3 are being protected by the fourth mask layer MSK4, the fourth probe solution is not coated onto the pixels PX in the first assay A1, the second assay A2, and the third assay A3. In some embodiments, since the sensing layer 160 is being activated in the step shown in FIG. 5A, the fourth probe 300d in the fourth probe solution is bonded to the sensing layer 160 of the pixels PX in the fourth assay A4. Thereafter, a suction process is performed on the fourth probe solution to remove the fourth solvent, thereby allowing the fourth probe 300d to be coated onto the pixels PX in the fourth assay A4. It should be noted that since the fourth probe 300d is bonded to the sensing layer 160 of the pixels PX in the fourth assay A4, the suction process would only remove the fourth solvent and would not remove the fourth probe 300d. After the fourth probe 300d is coated onto the pixels PX in the fourth assay A4, the fourth mask layer MSK4 is removed.

Referring to FIG. 5F, by performing the steps shown in FIG. 5A to FIG. 5E, pixels PX in different assays of the sensor array SA may be coated with different probes. For example, the pixels PX in the first assay A1 is coated with the first probe 300a, the pixels PX in the second assay A2 is coated with the second probe 300b, the pixels PX in the third assay A3 is coated with the third probe 300c, and the pixels PX in the fourth assay A4 is coated with the fourth probe 300d. Please be noted that although the coating process in FIG. 5A to FIG. 5F is performed on a chip (the bio-sensing integrated circuit 100), the disclosure is not limited thereto. In some alternative embodiments, the coating process in FIG. 5A to FIG. 5E may be applicable to other types of medium, such as a printed circuit board (PCB) or the like.

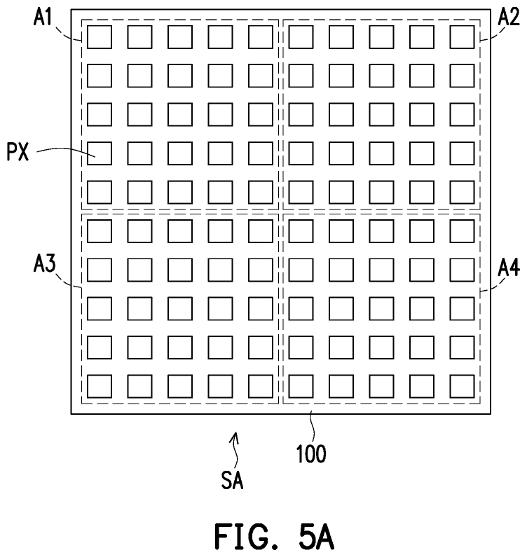

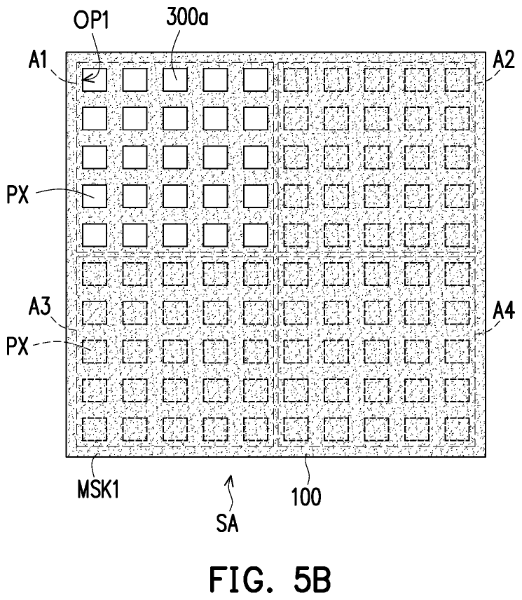

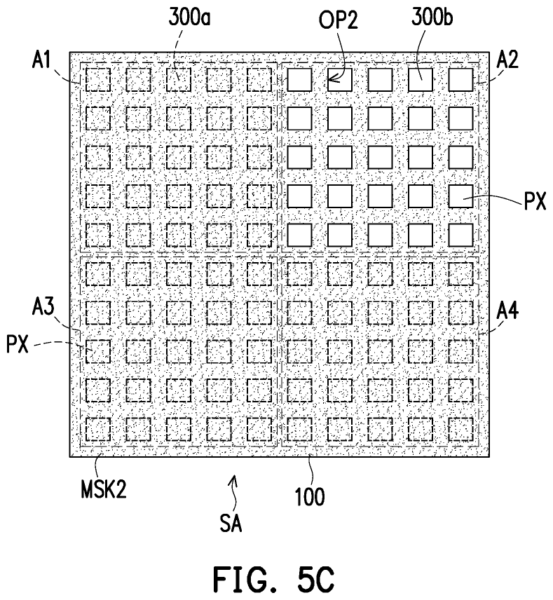

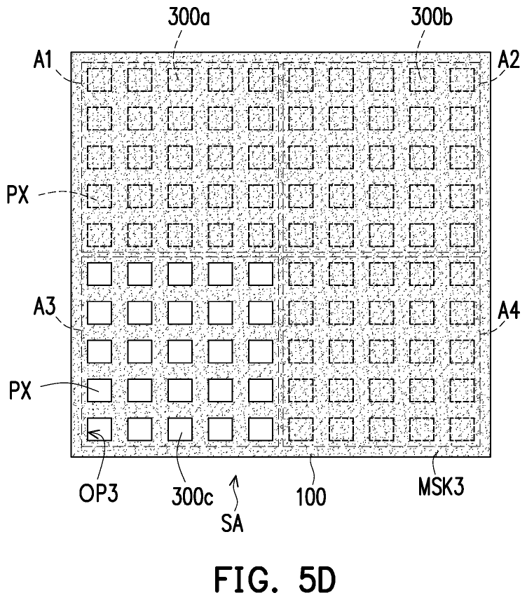

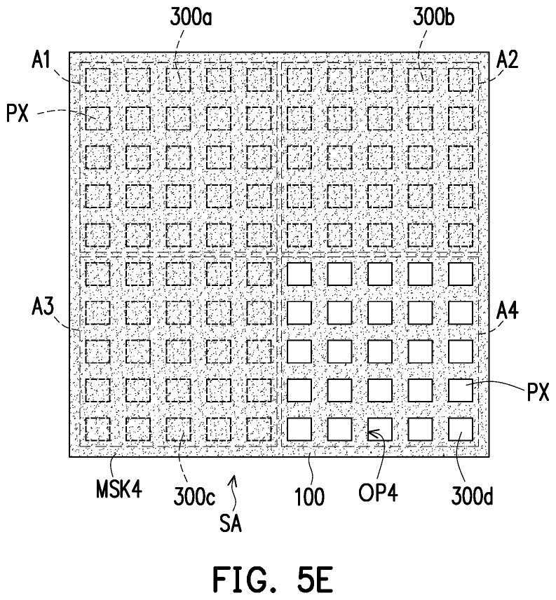

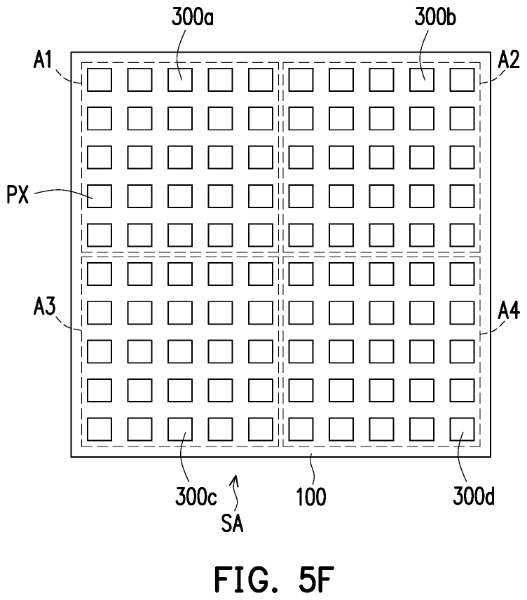

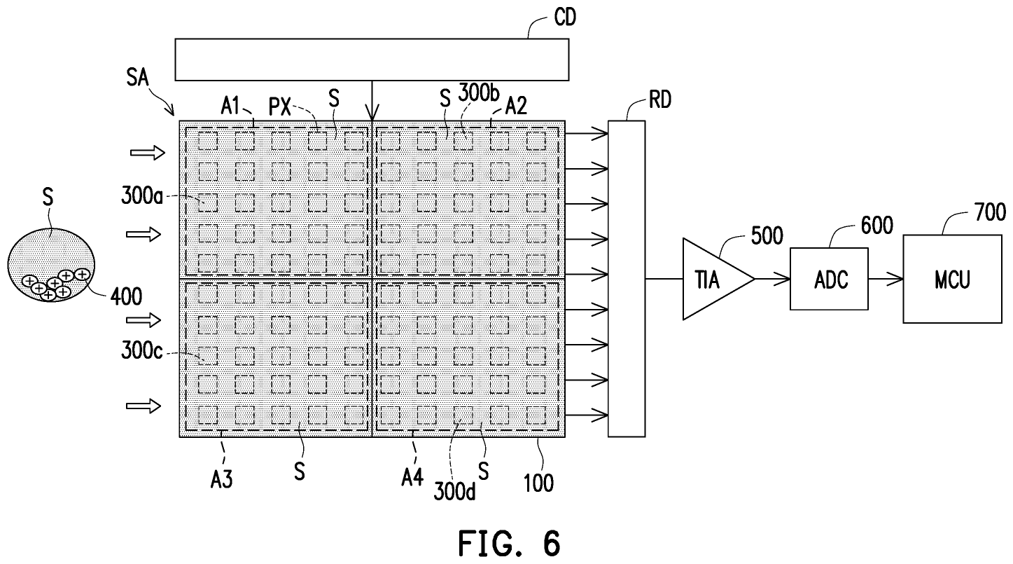

In some embodiments, the sensor array SA coated with different probes as shown in FIG. 5F may be used in the target material identification method. The target material identification method will be exemplified in detail below in conjunction with FIG. 6, FIG. 7, FIG. 8, FIGS. 9A-9D, FIG. 10, FIG. 11, FIG. 12, and FIGS. 13A-13D.

FIG. 6 is a schematic flow of a target material identification method in accordance with some embodiments of the disclosure. Referring to FIG. 6, the bio-sensing integrated circuit 100 having the sensor array SA in FIG. 5F is provided. In some embodiments, the pixels PX in the first assay A1 is coated with the first probe 300a, the pixels PX in the second assay A2 is coated with the second probe 300b, the pixels PX in the third assay A3 is coated with the third probe 300c, and the pixels PX in the fourth assay A4 is coated with the fourth probe 300d.

In some embodiments, a calibration process is performed on the sensor array SA of the bio-sensing integrated circuit 100. For example, the calibration process is performed to measure the current between the source region 144 and the drain region 146 in each pixel PX. In some embodiments, the column decoder CD provides a column selection signal to the pixels PX in the sensor array SA and the row decoder RD provides a row selection signal to the pixels PX in the sensor array SA. For example, based on the column selection signal and the row selection signal, the BioFET 140 in the selected pixel PX is turned on, and the current between the source region 144 and the drain region 146 of the BioFET 140 in the selected pixel PX is measured. Thereafter, the current between the source region 144 and the drain region 146 measured for each pixel PX is transmitted to the TIA 500 in a form of an analog signal. The TIA 500 then enhances and magnifies the analog signal received. Subsequently, the analog signal leaves the TIA 500 and is transmitted to an analog-to-digital converter (ADC) 600. The ADC 600 converts the signal received from an analog signal to a digital signal, and outputs the digital signal to a microcontroller unit (MCU) 700. In some embodiments, the MCU 700 processes the digital signal received by a software or the like. In other words, the digital signal received by MCU 700 may be standardized before being outputted. For example, an average of the currents between the source region 144 and the drain region 146 in the pixels PX of the same assay may be calculated, and the result outputted corresponds to this average value. However, the disclosure is not limited thereto. In some alternative embodiments, the digital signal received by MCU 700 may be standardized through other means. After the digital signal is being processed, the MCU 700 outputs the currents between the source region 144 and the drain region 146 for the pixels PX in the first assay A1, the pixels PX in the second assay A2, the pixels PX in the third assay A3, and the pixels PX in the fourth assay A4 as a function of time.

In some embodiments, the measurement of the pixels PX in the first assay A1, the pixels PX in the second assay A2, the pixels PX in the third assay A3, and the pixels PX in the fourth assay A4 are taken place in sequential order. For example, the MCU 700 outputs the currents between the source region 144 and the drain region 146 for the pixels PX in the first assay A1, the pixels PX in the second assay A2, the pixels PX in the third assay A3, and the pixels PX in the fourth assay A4 in sequential order. That is, the calibration process may be divided into a first calibration process for the pixels PX in the first assay A1, a second calibration process for the pixels PX in the second assay A2, a third calibration process for the pixels PX in the third assay A3, and a fourth calibration process for the pixels PX in the fourth assay A4, and these calibration processes are performed in sequential order. In some embodiments, the results for the pixels PX in the first assay A1 is referred to as a 1^{st} pre-test measurement value, the results for the pixels PX in the second assay A2 is referred to as a 2^{nd} pre-test measurement value, the results for the pixels PX in the third assay A3 is referred to as a 3^{rd} pre-test measurement value, and the results for the pixels PX in the fourth assay A4 is referred to as a 4th pre-test measurement value. In some embodiments, the results are shown in FIG. 7. In some embodiments, a limit of detection (LoD) of the bio-sensing integrated circuit 100 may also be determined during the calibration process. In some embodiments, the LoD ranges from about 0.1 fg/mL to about 1000 fg/mL.

After the calibration process is completed, a sample fluid S having a target material 400 therein is provided. Then, a bio-sensing process may be performed. First, the sample fluid S is applied to the sensor array SA of the bio-sensing integrated circuit 100. In some embodiments, the sample fluid S is applied to the first assay A1, the second assay A2, the third assay A3, and the fourth assay A4 at once. Then, the sensor array SA with the sample fluid S applied thereon is allowed to sit still for a certain period for incubation. Thereafter, a washing step is performed to remove the excessive sample fluid S. In some embodiments, the washing step may be performed by using automatic pump, so as to ensure minimum variation between different assays. As mentioned above, depending on the type of the probe (i.e. the first probe 300a, the second probe 300b, the third probe 300c, or the fourth probe 300d) coated on the sensor array SA, the target material 400 in the sample fluid S may or may not bind to the first probe 300a, the second probe 300b, the third probe 300c, and the fourth probe 300d in the respective sensing well SW (shown in FIGS. 1 and 2). In some embodiments, the binding between the target material 400 and the probe (i.e. the first probe 300a, the second probe 300b, the third probe 300c, or the fourth probe 300d) would alter the conductance of the channel region 148 underneath the sensing well SW (shown in FIG. 1). This change in conductance would affect the current between the source region 144 and the drain region 146, so measuring the current between the source region 144 and the drain region 146 in each pixel PX allows the determination of the binding of the target material 400. Similar to that of the calibration process, the currents between the source region 144 and the drain region 146 of the pixels PX are measured and outputted with the aid of the column decoder CD, the row decoder RD, the TIA 500, the ADC 600, and the MCU 700. In some embodiments, the measurement of the pixels PX in the first assay A1, the pixels PX in the second assay A2, the pixels PX in the third assay A3, and the pixels PX in the fourth assay A4 are taken place in sequential order.

For example, the MCU 700 outputs the currents between the source region 144 and the drain region 146 for the pixels PX in the first assay A1, the pixels PX in the second assay A2, the pixels PX in the third assay A3, and the pixels PX in the fourth assay A4 in sequential order. That is, the bio-sensing process may be divided into a first bio-sensing process for the pixels PX in the first assay A1, a second bio-sensing process for the pixels PX in the second assay A2, a third bio-sensing process for the pixels PX in the third assay A3, and a fourth bio-sensing process for the pixels PX in the fourth assay A4, and these bio-sensing processes are performed in sequential order. In some embodiments, the result for the pixels PX in the first assay A1 is referred to as a 1^{st} post-test measurement value, the result for the pixels PX in the second assay A2 is referred to as a 2^{nd} post-test measurement value, the result for the pixels PX in the third assay A3 is referred to as a 3rd post-test measurement value, and the result for the pixels PX in the fourth assay A4 is referred to as a 4^{th} post-test measurement value. In some embodiments, the results are shown in FIG. 7.

In some embodiments, by comparing the pre-test measurement values and the corresponding post-test measurement values, whether the target material 400 is bind to the probe in a certain assay may be determined. For example, the pre-test measurement value in a certain assay may be compared with the corresponding post-test measurement value in the same assay to determine whether there is a different between the two. If there is no significant difference between the two, the target material 400 is not bind to the probe coated in this assay. If there is a significant difference between the two, the target material 400 is bind to the probe coated in this assay, and this assay is marked as a binding assay. Then, the target material 400 may be identified based on the probe in the binding assay. The determination of whether the target material 400 is bind to the probe in a certain assay will be exemplified below in conjunction with FIG. 7.

FIG. 7 is a current vs. time curve of the sample fluid S in the target material identification method of FIG. 6. In FIG. 7, the time period between t₀ and t₄ denotes a period before the bio-sensing process, the time period between t₄ and t₅ denotes a period during the bio-sensing process (for example, the incubation period), and the time period between t₅ and t₉ denotes a period after the bio-sensing process. On the other hand, the measurement between t₀ and t₁ corresponds to the 1^{st} pre-test measurement value for the first assay A1, the measurement between t₁ and t₂ corresponds to the 2^{nd} pre-test measurement value for the second assay A2, the measurement between t₂ and t₃ corresponds to the 3^{rd} pre-test measurement value for the third assay A3, the measurement between t₃ and t₄ corresponds to the 4^{th} pre-test measurement value for the fourth assay A4, the measurement between t₅ and t₆ corresponds to the 1^{st} post-test measurement value for the first assay A1, the measurement between t₆ and t₇ corresponds to the 2^{nd} post-test measurement value for the second assay A2, the measurement between t₇ and t₈ corresponds to the 3rd post-test measurement value for the third assay A3, and the measurement between t₈ and t₉ corresponds to the 4^{th} post-test measurement value for the fourth assay A4.

As illustrated in FIG. 7, there is a significant difference between the 1^{st} pre-test measurement value (i.e. the measurement between t₀ and t₁) and the 1^{st} post-test measurement value (i.e. the measurement between t₅ and t₆). Therefore, the target material 400 is bind to the first probe 300a in the first assay A1, and the first assay A1 is marked as a binding assay. On the other hand, there is no significant different between the 2^{nd} pre-test measurement value (i.e. the measurement between t₁ and t₂) and the 2^{nd} post-test measurement value (i.e. the measurement between t₆ and t₇), between the 3^{rd} pre-test measurement value (i.e. the measurement between t₂ and t₃) and the 3rd post-test measurement value (i.e. the measurement between t₇ and t₈), and between the 4^{th} pre-test measurement value (i.e. the measurement between t₃ and t₄) and the 4^{th} post-test measurement value (i.e. the measurement between t₈ and t₉), so the target material 400 is not bind to the second probe 300b in the second assay A2, the third probe 300c in the third assay A3, and the fourth probe 300d in the fourth assay A4. Please be noted that the slight differences between the 2^{nd} pre-test measurement value and the 2^{nd} post-test measurement value, between the 3^{rd} pre-test measurement value and the 3rd post-test measurement value, and between the 4^{th} pre-test measurement value and the 4^{th} post-test measurement value are originated from noise or marginal error (derived from the washing step or other factors), and can be negligible. Since the first assay A1 is being marked as the binding assay, the type of the target material 400 may be identified based on the type of the first probe 300a.

In some embodiments, by utilizing the sensor array SA with various assays (i.e. the first assay A1, the second assay A2, the third assay A3, and the fourth assay A4) at once, one time test may be performed. As such, the testing efficiency may be sufficiently enhanced. For example, the experimental time may be reduced to 15 minutes or less.

Please be noted that although the target material identification method shown in FIG. 6 and FIG. 7 utilizes the sensor array SA with four assays A1-A4, the disclosure is not limited thereto. Depending on the number of different probes, the number of the assays in the sensor array SA may vary. For example, the number of the arrays may be ten, hundreds, thousands, or so as long as these arrays are all coated with different types of probes. In some embodiments, when the number of the assays is too many, one bio-sensing integrated circuit 100 may not be sufficient. As such, multiple bio-sensing integrated circuits 100 may be utilized. Meanwhile, the sensor array SA of each bio-sensing integrated circuit 100 is still being divided into multiple assays.

FIG. 8 is a schematic flow of a target material identification method in accordance with some alternative embodiments of the disclosure. Referring to FIG. 8, the target material identification method in FIG. 8 is similar to the target material identification method in FIG. 6, so similar elements are denoted by the same reference numeral and the detailed description thereof is omitted herein. However, in the target material identification method in FIG. 8, the measurement of the pixels PX in the first assay A1, the pixels PX in the second assay A2, the pixels PX in the third assay A3, and the pixels PX in the fourth assay A4 are conducted in parallel. For example, the first calibration process, the second calibration process, the third calibration process, and the fourth calibration process are performed simultaneously. Similarly, the first bio-sensing process, the second bio-sensing process, the third bio-sensing process, and the fourth bio-sensing process are also performed simultaneously.

As illustrated in FIG. 8, the current between the source region 144 and the drain region 146 measured for each pixel PX in the first assay A1 is transmitted to the first TIA 500a, the current between the source region 144 and the drain region 146 measured for each pixel PX in the second assay A2 is transmitted to the second TIA 500b, the current between the source region 144 and the drain region 146 measured for each pixel PX in the third assay A3 is transmitted to the third TIA 500c, and the current between the source region 144 and the drain region 146 measured for each pixel PX in the fourth assay A4 is transmitted to the fourth TIA 500d simultaneously. The first TIA 500a, the second TIA 500b, the third TIA 500c, and the fourth TIA 500d then enhance and magnify the analog signal received. Subsequently, the analog signals leave the first TIA 500a, the second TIA 500b, the third TIA 500c, and the fourth TIA 500d and are respectively transmitted to a first ADC 600a, a second ADC 600b, a third ADC 600c, and a fourth ADC 600d simultaneously. The first ADC 600a, the second ADC 600b, the third ADC 600c, and the fourth ADC 600d convert the signals received from analog signals to digital signals, and output the digital signals to the MCU 700 simultaneously. In some embodiments, the MCU 700 processes the digital signals received by a software or the like. In other words, the digital signals received by MCU 700 may be standardized before being outputted. For example, an average of the currents between the source region 144 and the drain region 146 in the pixels PX of the same assay may be calculated, and the result outputted corresponds to this average value. However, the disclosure is not limited thereto. In some alternative embodiments, the digital signal received by MCU 700 may be standardized through other means. After the digital signal is being processed, the MCU 700 outputs the currents between the source region 144 and the drain region 146 for the pixels PX in the first assay A1, the pixels PX in the second assay A2, the pixels PX in the third assay A3, and the pixels PX in the fourth assay A4 as a function of time. In some embodiments, the results are shown in FIG. 9A to FIG. 9D.

FIG. 9A is a current vs. time curve of the sample fluid S in the first assay A1 in the target material identification method of FIG. 8. FIG. 9B is a current vs. time curve of the sample fluid S in the second assay A2 in the target material identification method of FIG. 8. FIG. 9C is a current vs. time curve of the sample fluid S in the third assay A3 in the target material identification method of FIG. 8. FIG. 9D is a current vs. time curve of the sample fluid S in the fourth assay A4 in the target material identification method of FIG. 8. In FIG. 9A to FIG. 9D, the time period between t₀ and t₁ denotes a period before the bio-sensing process, the time period between t₁ and t₂ denotes a period during the bio-sensing process (for example, the incubation period), and the time period after t₂ denotes a period after the bio-sensing process.

As illustrated in FIG. 9A, there is a significant difference between the 1^{st} pre-test measurement value (i.e. the measurement between t₀ and t₁) and the 1^{st} post-test measurement value (i.e. the measurement after t₂). Therefore, the target material 400 is bind to the first probe 300a in the first assay A1, and the first assay A1 is marked as a binding assay. On the other hand, as illustrated in FIG. 9B to FIG. 9D, there is no significant different between the 2^{nd} pre-test measurement value (i.e. the measurement between t₁ and t₂ in FIG. 9B) and the 2^{nd} post-test measurement value (i.e. the measurement after t₂ in FIG. 9B), between the 3^{rd} pre-test measurement value (i.e. the measurement between t₁ and t₂ in FIG. 9C) and the 3rd post-test measurement value (i.e. the measurement after t₂ in FIG. 9C), and between the 4^{th} pre-test measurement value (i.e. the measurement between t₁ and t₂ in FIG. 9D) and the 4^{th} post-test measurement value (i.e. the measurement after t₂ in FIG. 9D), so the target material 400 is not bind to the second probe 300b in the second assay A2, the third probe 300c in the third assay A3, and the fourth probe 300d in the fourth assay A4. Please be noted that the slight differences between the 2^{nd} pre-test measurement value and the 2^{nd} post-test measurement value, between the 3^{rd} pre-test measurement value and the 3rd post-test measurement value, and between the 4^{th} pre-test measurement value and the 4^{th} post-test measurement value are originated from noise or marginal error (derived from the washing step or other factors), and can be negligible. Since the first assay A1 is being marked as the binding assay, the type of the target material 400 may be identified based on the type of the first probe 300a.

In some embodiments, by utilizing the sensor array SA with various assays (i.e. the first assay A1, the second assay A2, the third assay A3, and the fourth assay A4) at once, one time test may be performed. As such, the testing efficiency may be sufficiently enhanced. For example, the experimental time may be reduced to 15 minutes or less.

Please be noted that although the target material identification method shown in FIG. 8 and FIG. 9A to FIG. 9D utilizes the sensor array SA with four assays A1-A4, the disclosure is not limited thereto. Depending on the number of different probes, the number of the assays in the sensor array SA may vary. For example, the number of the arrays may be ten, hundreds, thousands, or so as long as these arrays are all coated with different types of probes. In some embodiments, when the number of the assays is too many, one bio-sensing integrated circuit 100 may not be sufficient. As such, multiple bio-sensing integrated circuits 100 may be utilized. Meanwhile, the sensor array SA of each bio-sensing integrated circuit 100 is still being divided into multiple assays.

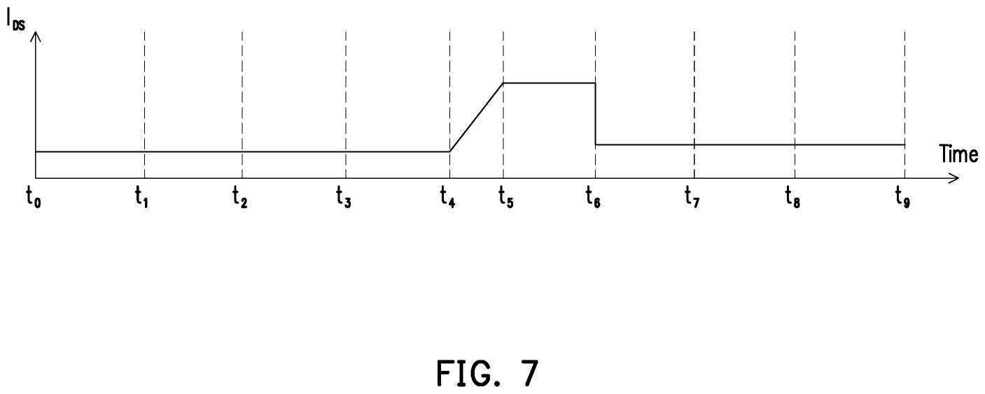

FIG. 10 is a schematic flow of a target material identification method in accordance with some alternative embodiments of the disclosure. Referring to FIG. 10, a first bio-sensing integrated circuit 100a having a first sensor array SA1, a second bio-sensing integrated circuit 100b having a second sensor array SA2, a third bio-sensing integrated circuit 100c having a third sensor array SA3, and a fourth bio-sensing integrated circuit 100d having a fourth sensor array SA4 are provided. In some embodiments, the first bio-sensing integrated circuit 100a, the second bio-sensing integrated circuit 100b, the third bio-sensing integrated circuit 100c, and the fourth bio-sensing integrated circuit 100d are identical to one another and may be similar to the bio-sensing integrated circuit 100 in FIG. 1 and FIG. 4, so the detailed descriptions thereof are omitted herein. However, the first bio-sensing integrated circuit 100a, the second bio-sensing integrated circuit 100b, the third bio-sensing integrated circuit 100c, and the fourth bio-sensing integrated circuit 100d are respectively coated with different types of probes. In some embodiments, the first bio-sensing integrated circuit 100a, the second bio-sensing integrated circuit 100b, the third bio-sensing integrated circuit 100c, and the fourth bio-sensing integrated circuit 100d may be placed on a same cartridge. In some embodiments, the first sensor array SA1 correspond to a first assay A1, the second sensor array SA2 corresponds to a second assay A2, the third sensor array SA3 corresponds to a third assay A3, and the fourth sensor array SA4 corresponds to a fourth assay A4. In some embodiments, the first assay A1 corresponds to multiple first pixels PX1, the second assay A2 correspond to multiple second pixels PX2, the third assay A3 corresponds to multiple third pixels PX3, and the fourth assay A4 corresponds to multiple fourth pixels PX4, so as to ensure the detection precision. As illustrated in FIG. 10, the first pixels PX1 of the first bio-sensing integrated circuit 100a are coated with a first probe 300a, the second pixels PX2 of the second-bio integrated circuit 100b are coated with a second probe 300b, the third pixels PX3 of the third bio-integrated circuit 100c are coated with a third probe 300c, and the fourth pixels PX4 of the fourth bio-integrated circuit 100d are coated with a fourth probe 300d.

In some embodiments, a calibration process is performed on the first sensor array SA1 of the first bio-sensing integrated circuit 100a, the second sensor array SA2 of the second bio-sensing integrated circuit 100b, the third sensor array SA3 of the third bio-sensing integrated circuit 100c, and the fourth sensor array SA4 of the fourth bio-sensing integrated circuit 100d. For example, the calibration process is performed to measure the current between the source region 144 and the drain region 146 in each pixel (i.e. the first pixel PX1, the second pixel PX2, the third pixel PX3, and the fourth pixel PX4). In some embodiments, a first column decoder CD1 provides a column selection signal to the first pixels PX1 in the first sensor array SA1 and a first row decoder RD1 provides a row selection signal to the first pixels PX1 in the first sensor array SA1. A second column decoder CD2 provides a column selection signal to the second pixels PX2 in the second sensor array SA2 and a second row decoder RD2 provides a row selection signal to the second pixels PX2 in the second sensor array SA2. A third column decoder CD3 provides a column selection signal to the third pixels PX3 in the third sensor array SA3 and a third row decoder RD3 provides a row selection signal to the third pixels PX3 in the third sensor array SA3. A fourth column decoder CD4 provides a column selection signal to the fourth pixels PX4 in a fourth sensor array SA4 and the fourth row decoder RD4 provides a row selection signal to the fourth pixels PX4 in the fourth sensor array SA4. For example, based on the column selection signal and the row selection signal, the BioFET 140 in the selected pixel (i.e. the first pixel PX1, the second pixel PX2, the third pixel PX3, or the fourth pixel PX4) is turned on, and the current between the source region 144 and the drain region 146 of the BioFET 140 in the selected pixel is measured. Thereafter, the currents between the source region 144 and the drain region 146 measured for each first pixel PX1, each second pixel PX2, each third pixel PX3, and each fourth pixel PX4 are transmitted to a multiplexer (MUX) 800 in a form of analog signals. The MUX 800 then selects a particular analog signal received and forwards the selected signal to the TIA 500. The TIA 500 enhances and magnifies the analog signal received. Subsequently, the analog signal leaves the TIA 500 and is transmitted to an analog-to-digital converter (ADC) 600. The ADC 600 converts the signal received from an analog signal to a digital signal, and outputs the digital signal to a microcontroller unit (MCU) 700. In some embodiments, the MCU 700 processes the digital signal received by a software or the like. In other words, the digital signal received by MCU 700 may be standardized before being outputted. For example, an average of the currents between the source region 144 and the drain region 146 in the first pixels PX1, an average of the currents between the source region 144 and the drain region 146 in the second pixels PX2, an average of the currents between the source region 144 and the drain region 146 in the third pixels PX3, and an average of the currents between the source region 144 and the drain region 146 in the fourth pixels PX4 may be independently calculated, and the results outputted correspond to these average values. However, the disclosure is not limited thereto. In some alternative embodiments, the digital signal received by MCU 700 may be standardized through other means. After the digital signal is being processed, the MCU 700 outputs the currents between the source region 144 and the drain region 146 for the first pixels PX1 in the first sensor array SA1, the second pixels PX2 in the second sensor array SA2, the third pixels PX3 in the third sensor array SA3, and the fourth pixels PX4 in the fourth sensor array SA4 as a function of time.

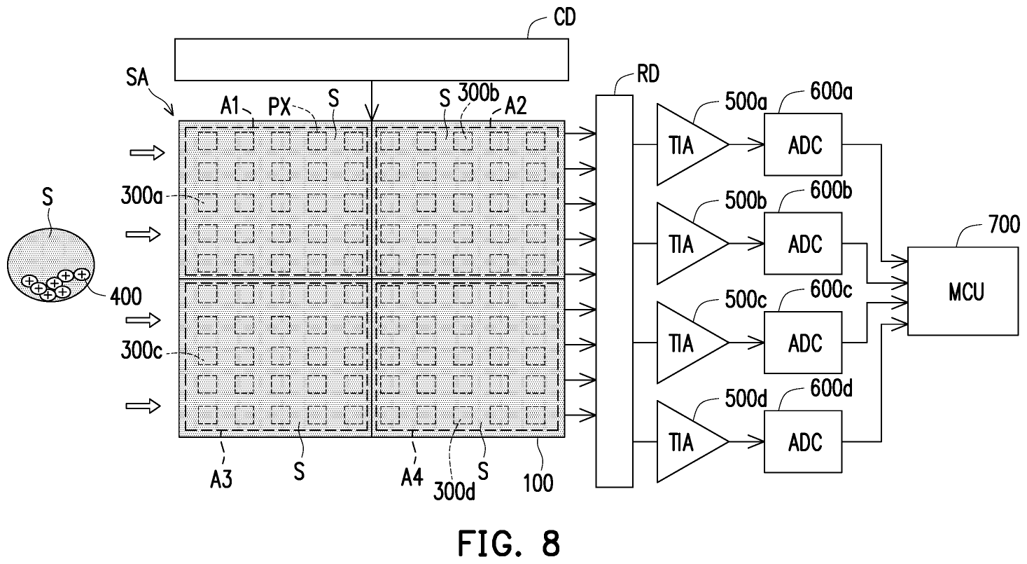

In some embodiments, the measurement of the first pixels PX1 in the first sensor array SA1, the second pixels PX2 in the second sensor array SA2, the third pixels PX3 in the third sensor array SA3, and the fourth pixels PX4 in the fourth sensor array SA4 are taken place in sequential order. For example, after the currents between the source region 144 and the drain region 146 for the first pixels PX1 in the first sensor array SA1 are being processed by the MUX 800, the TIA 500, the ADC 600, and the MCU 700, the MCU 700 commands the MUX 800 to process the currents between the source region 144 and the drain region 146 for the second pixels PX2 in the second sensor array SA2, as denoted by the arrow between the MCU 700 and the MUX 800. Similarly, after the currents between the source region 144 and the drain region 146 for the second pixels PX2 in the second sensor array SA2 are being processed by the MUX 800, the TIA 500, the ADC 600, and the MCU 700, the MCU 700 commands the MUX 800 to process the currents between the source region 144 and the drain region 146 for the third pixels PX3 in the third sensor array SA3. Then, after the currents between the source region 144 and the drain region 146 for the third pixels PX3 in the third sensor array SA3 are being processed by the MUX 800, the TIA 500, the ADC 600, and the MCU 700, the MCU 700 commands the MUX 800 to process the currents between the source region 144 and the drain region 146 for the fourth pixels PX4 in the fourth sensor array SA4. In some embodiments, the MCU 700 outputs the currents between the source region 144 and the drain region 146 for the first pixels PX1 in the first sensor array SA1, the second pixels PX2 in the second sensor array SA2, the third pixels PX3 in the third sensor array SA3, and the fourth pixels PX4 in the fourth sensor array SA4 in sequential order. That is, the calibration process may be divided into a first calibration process for the first pixels PX1 in the first sensor array SA1, a second calibration process for the second pixels PX2 in the second sensor array SA2, a third calibration process for the third pixels PX3 in the third sensor array SA3, and a fourth calibration process for the fourth pixels PX4 in the fourth sensor array SA4, and these calibration processes are performed in sequential order. In some embodiments, the result for the first pixels PX1 in the first sensor array SA1 is referred to as a 1^{st} pre-test measurement value, the result for the second pixels PX2 in the second sensor array SA2 is referred to as a 2^{nd} pre-test measurement value, the result for the third pixels PX3 in the third sensor array SA3 is referred to as a 3^{rd} pre-test measurement value, and the result for the fourth pixels PX4 in the fourth sensor array SA4 is referred to as a 4^{th} pre-test measurement value. In some embodiments, the results are shown in FIG. 11. In some embodiments, a limit of detection (LoD) of the first bio-sensing integrated circuit 100a, the second bio-sensing integrated circuit 100b, the third bio-sensing integrated circuit 100c, and the fourth bio-sensing integrated circuit 100d may also be determined during the calibration process. In some embodiments, the LoD ranges from about 0.1 fg/mL to about 1000 fg/mL.

After the calibration process is completed, a sample fluid S having a target material 400 therein is provided. Then, a bio-sensing process may be performed. First, the sample fluid S is applied to the first sensor array SA1 of the first bio-sensing integrated circuit 100a, the second sensor array SA2 of the second bio-sensing integrated circuit 100b, the third sensor array SA3 of the third bio-sensing integrated circuit 100c, and the fourth sensor SA4 of the fourth bio-sensing integrated circuit 100d. In some embodiments, the sample fluid S is applied to the first assay A1 (i.e. the first sensor array SA1), the second assay A2 (i.e. the second sensor array A2), the third assay A3 (i.e. the third sensor array A3), and the fourth assay A4 (i.e. the fourth sensor array A4) at once. Then, the first sensor array SA1, the second sensor array SA2, the third sensor array SA3, and the fourth sensor array SA4 with the sample fluid S applied thereon are allowed to sit still for a certain period for incubation. Thereafter, a washing step is performed to remove the excessive sample fluid S. In some embodiments, the washing step may be performed by using automatic pump, so as to ensure minimum variation between different assays. As mentioned above, depending on the type of the probe (i.e. the first probe 300a, the second probe 300b, the third probe 300c, or the fourth probe 300d) coated on the sensor arrays, the target material 400 in the sample fluid S may or may not bind to the first probe 300a, the second probe 300b, the third probe 300c, and the fourth probe 300d in the respective sensing well SW (shown in FIGS. 1 and 2). In some embodiments, the binding between the target material 400 and the probe (i.e. the first probe 300a, the second probe 300b, the third probe 300c, or the fourth probe 300d) would alter the conductance of the channel region 148 underneath the sensing well SW (shown in FIG. 1). This change in conductance would affect the current between the source region 144 and the drain region 146, so measuring the current between the source region 144 and the drain region 146 in each first pixel PX1, each second pixel PX2, each third pixel PX3, and each fourth pixel PX4 allows the determination of the binding of the target material 400. Similar to that of the calibration process, the currents between the source region 144 and the drain region 146 of the first pixels PX1, the second pixels PX2, the third pixels PX3, and the fourth pixels PX4 are measured and outputted with the aid of the first column decoder CD1, the second column decoder CD2, the third column decoder CD3, the fourth column decoder CD4, the first row decoder RD1, the second row decoder RD2, the third row decoder RD3, the fourth row decoder RD4, the TIA 500, the ADC 600, the MCU 700, and the MUX 800. In some embodiments, the measurement of the first pixels PX1 in the first sensor array SA1, the second pixels PX2 in the second sensor array SA2, the third pixels PX3 in the third sensor array SA3, and the fourth pixels PX4 in the fourth sensor array SA4 are taken place in sequential order. For example, the MCU 700 outputs the currents between the source region 144 and the drain region 146 for the first pixels PX1 in the first sensor array SA1, the second pixels PX2 in the second sensor array SA2, the third pixels PX3 in the third sensor array SA3, and the fourth pixels PX4 in the fourth sensor array SA4 in sequential order. That is, the bio-sensing process may be divided into a first bio-sensing process for the first pixels PX1 in the first sensor array SA1, a second bio-sensing process for the second pixels PX2 in the second sensor array SA2, a third bio-sensing process for the third pixels PX3 in the third sensor array SA3, and a fourth bio-sensing process for the fourth pixels PX4 in the fourth sensor array SA4, and these bio-sensing processes are performed in sequential order. In some embodiments, the result for the first pixels PX1 in the first sensor array SA1 is referred to as a 1^{st} post-test measurement value, the result for the second pixels PX2 in the second sensor array SA2 is referred to as a 2^{nd} post-test measurement value, the result for the third pixels PX3 in the third sensor array SA3 is referred to as a 3^{rd} post-test measurement value, and the result for the fourth pixels PX4 in the fourth sensor array SA4 is referred to as a 4^{th} post-test measurement value. In some embodiments, the results are shown in FIG. 11.

In some embodiments, by comparing the pre-test measurement values and the corresponding post-test measurement values, whether the target material 400 is bind to the probe in a certain sensor array may be determined. For example, the pre-test measurement value in a certain sensor array may be compared with the corresponding post-test measurement value in the same sensor array to determine whether there is a different between the two. If there is no significant difference between the two, the target material 400 is not bind to the probe coated in this sensor array. If there is a significant difference between the two, the target material 400 is bind to the probe coated in this sensor array, and this sensor array is marked as a binding sensor array. Then, the target material 400 may be identified based on the probe in the binding sensor array. The determination of whether the target material 400 is bind to the probe in a certain sensor array will be exemplified below in conjunction with FIG. 11.

FIG. 11 is a current vs. time curve of the sample fluid in the target material identification method of FIG. 10. In FIG. 11, the time period between t₀ and t₄ denotes a period before the bio-sensing process, the time period between t₄ and t₅ denotes a period during the bio-sensing process (for example, the incubation period), and the time period between t₅ and t₉ denotes a period after the bio-sensing process. On the other hand, the measurement between t₀ and t₁ corresponds to the 1^{st} pre-test measurement value for the first sensor array SA1, the measurement between t₁ and t₂ corresponds to the 2^{nd} pre-test measurement value for the second sensor array SA2, the measurement between t₂ and t₃ corresponds to the 3^{rd} pre-test measurement value for the third sensor array SA3, the measurement between t₃ and t₄ corresponds to the 4^{th} pre-test measurement value for the fourth sensor array SA4, the measurement between t₅ and t₆ corresponds to the 1^{st} post-test measurement value for the first sensor array SA1, the measurement between t₆ and t₇ corresponds to the 2^{nd} post-test measurement value for the second sensor array SA2, the measurement between t₇ and t₈ corresponds to the 3^{rd} post-test measurement value for the third sensor array SA3, and the measurement between t₈ and t₉ corresponds to the 4^{th} post-test measurement value for the fourth sensor array SA4.

As illustrated in FIG. 11, there is a significant difference between the 1^{st} pre-test measurement value (i.e. the measurement between t₀ and t₁) and the 1^{st} post-test measurement value (i.e. the measurement between t₅ and t₆). Therefore, the target material 400 is bind to the first probe 300a in the first sensor array SA1, and the first sensor array SA1 is marked as a binding assay. On the other hand, there is no significant different between the 2^{nd} pre-test measurement value (i.e. the measurement between t₁ and t₂) and the 2^{nd} post-test measurement value (i.e. the measurement between t₆ and t₇), between the 3^{rd} pre-test measurement value (i.e. the measurement between t₂ and t₃) and the 3^{rd} post-test measurement value (i.e. the measurement between t₇ and t₈), and between the 4^{th} pre-test measurement value (i.e. the measurement between t₃ and t₄) and the 4^{th} post-test measurement value (i.e. the measurement between t₈ and t₉), so the target material 400 is not bind to the second probe 300b in the second sensor array SA2, the third probe 300c in the third sensor array SA3, and the fourth probe 300d in the fourth sensor array SA4. Please be noted that the slight differences between the 2^{nd} pre-test measurement value and the 2^{nd} post-test measurement value, between the 3^{rd} pre-test measurement value and the 3^{rd} post-test measurement value, and between the 4^{th} pre-test measurement value and the 4^{th} post-test measurement value are originated from noise or marginal error (derived from the washing step or other factors), and can be negligible. Since the first sensor array SA1 is being marked as the binding sensor array, the type of the target material 400 may be identified based on the type of the first probe 300a.

In some embodiments, by utilizing different sensor arrays (i.e. the first sensor array SA1, the second sensor array SA2, the third sensor array SA3, and the fourth sensor array SA4) with various assays (i.e. the first assay A1, the second assay A2, the third assay A3, and the fourth assay A4) at once, one time test may be performed. As such, the testing efficiency may be sufficiently enhanced. For example, the experimental time may be reduced to 15 minutes or less.

Please be noted that although the target material identification method shown in FIG. 10 and FIG. 11 utilizes four sensor arrays SA1-SA4 with four assays A1-A4, the disclosure is not limited thereto. Depending on the number of different probes, the number of the assays/the sensor arrays may vary. For example, the number of the assays/the sensor arrays may be ten, hundreds, thousands, or so as long as these assays/sensor arrays are all coated with different types of probes.

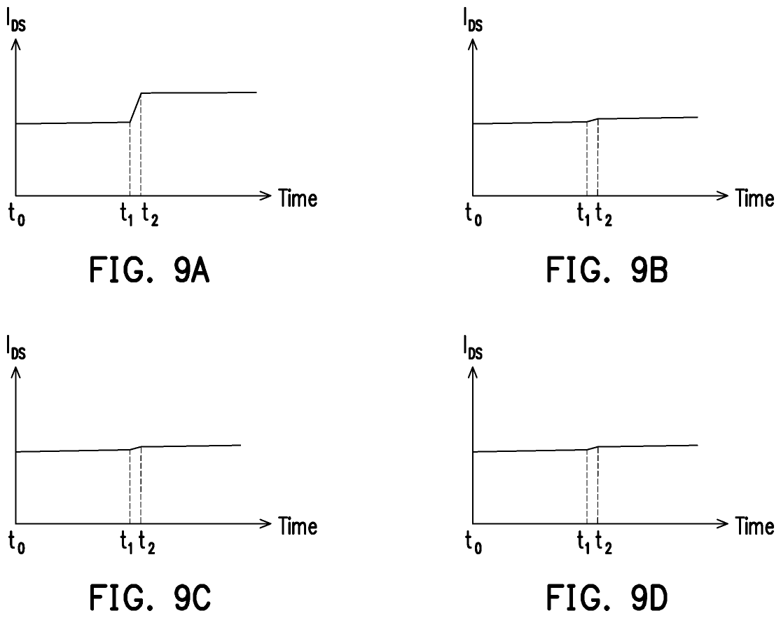

FIG. 12 is a schematic flow of a target material identification method in accordance with some alternative embodiments of the disclosure. Referring to FIG. 12, the target material identification method in FIG. 12 is similar to the target material identification method in FIG. 10, so similar elements are denoted by the same reference numeral and the detailed description thereof is omitted herein. However, the MUX 800 in FIG. 10 is omitted in the target material identification method in FIG. 12. In addition, in the target material identification method in FIG. 12, the measurement of the first pixels PX1 in the first sensor array SA1, the second pixels PX2 in the second sensor array SA2, the third pixels PX3 in the third sensor array SA3, and the fourth pixels PX4 in the fourth sensor array SA4 are conducted in parallel. For example, the first calibration process, the second calibration process, the third calibration process, and the fourth calibration process are performed simultaneously. Similarly, the first bio-sensing process, the second bio-sensing process, the third bio-sensing process, and the fourth bio-sensing process are also performed simultaneously.

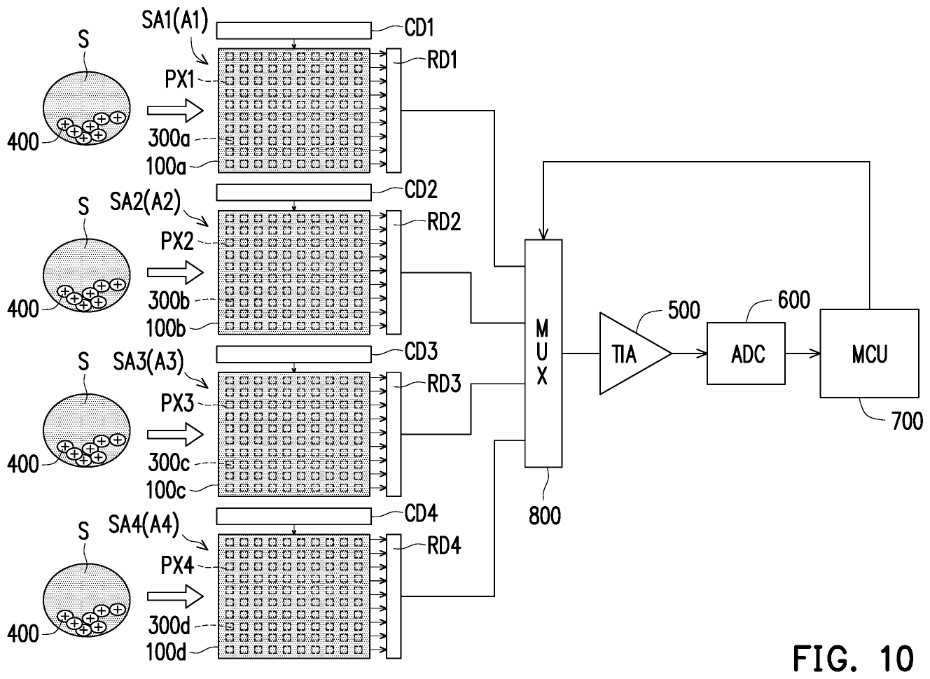

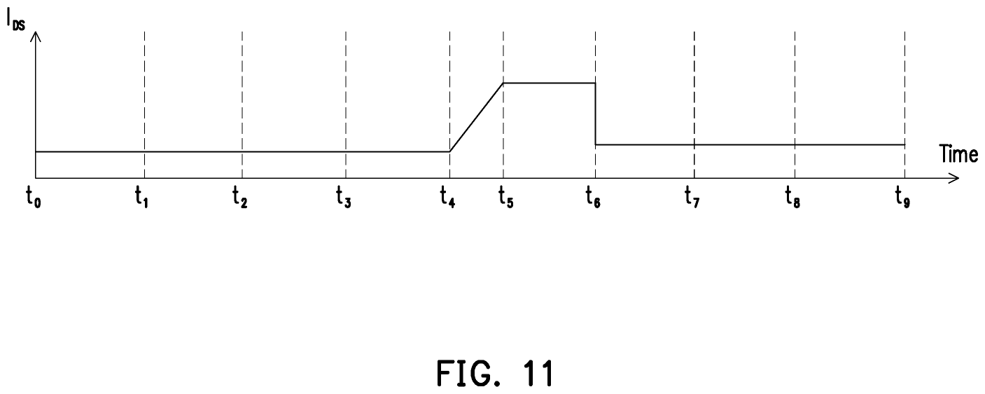

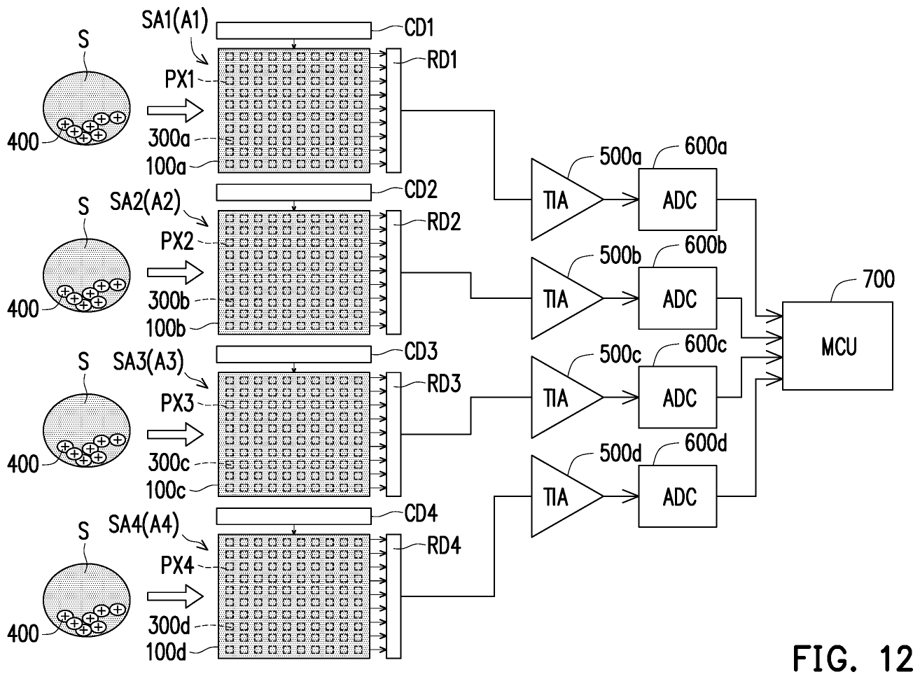

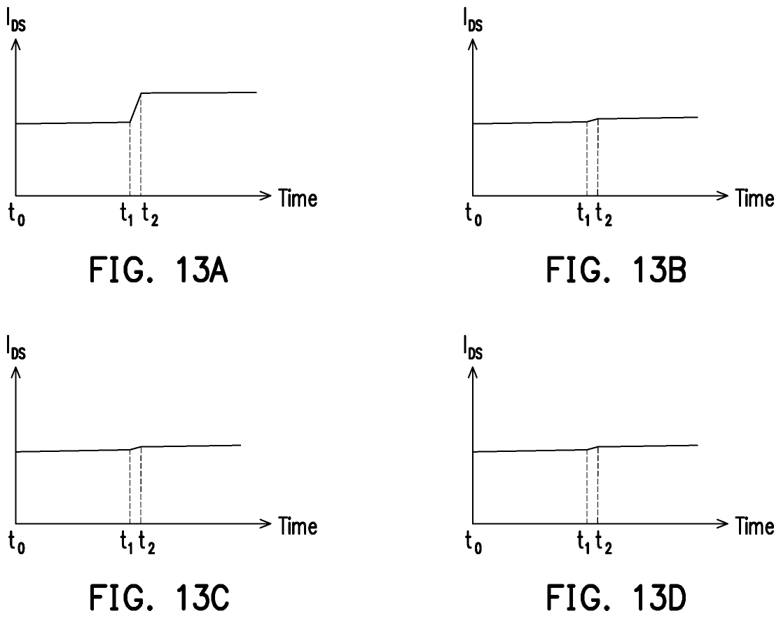

As illustrated in FIG. 12, the current between the source region 144 and the drain region 146 measured for each first pixel PX1 in the first sensor array SA1 is transmitted to the first TIA 500a, the current between the source region 144 and the drain region 146 measured for each second pixel PX2 in the second sensor array SA2 is transmitted to the second TIA 500b, the current between the source region 144 and the drain region 146 measured for each third pixel PX3 in the third sensor array SA3 is transmitted to the third TIA 500c, and the current between the source region 144 and the drain region 146 measured for each fourth pixel PX4 in the fourth sensor array SA4 is transmitted to the fourth TIA 500d simultaneously in a form of analog signals. The first TIA 500a, the second TIA 500b, the third TIA 500c, and the fourth TIA 500d then enhance and magnify the analog signals received. Subsequently, the analog signals leave the first TIA 500a, the second TIA 500b, the third TIA 500c, and the fourth TIA 500d and are respectively transmitted to a first ADC 600a, a second ADC 600b, a third ADC 600c, and a fourth ADC 600d simultaneously. The first ADC 600a, the second ADC 600b, the third ADC 600c, and the fourth ADC 600d convert the signals received from analog signals to digital signals, and output the digital signals to the MCU 700 simultaneously. In some embodiments, the MCU 700 processes the digital signals received by a software or the like. In other words, the digital signals received by MCU 700 may be standardized before being outputted. For example, an average of the currents between the source region 144 and the drain region 146 in the first pixels PX1, an average of the current between the source region 144 and the drain region 146 in the second pixels PX2, an average of the current between the source region 144 and the drain region 146 in the third pixels PX3, and an average of the current between the source region 144 and the drain region 146 in the fourth pixels PX4 may be independently calculated, and the results outputted correspond to these average values. However, the disclosure is not limited thereto. In some alternative embodiments, the digital signals received by MCU 700 may be standardized through other means. After the digital signals are being processed, the MCU 700 outputs the currents between the source region 144 and the drain region 146 for the first pixels PX1 in the first sensor array SA1, the second pixels PX2 in the second sensor array SA2, the third pixels PX3 in the third sensor array SA3, and the fourth pixels PX4 in the fourth sensor array SA4 as a function of time. In some embodiments, the results are shown in FIG. 13A to FIG. 13D.

FIG. 13A is a current vs. time curve of the sample fluid S in the first sensor array SA1 in the target material identification method of FIG. 12. FIG. 13B is a current vs. time curve of the sample fluid S in the second sensor array SA2 in the target material identification method of FIG. 12. FIG. 13C is a current vs. time curve of the sample fluid S in the third sensor array SA3 in the target material identification method of FIG. 12. FIG. 13D is a current vs. time curve of the sample fluid S in the fourth sensor array SA4 in the target material identification method of FIG. 12. In FIG. 13A to FIG. 13D, the time period between t₀ and t₁ denotes a period before the bio-sensing process, the time period between t₁ and t₂ denotes a period during the bio-sensing process (for example, the incubation period), and the time period after t₂ denotes a period after the bio-sensing process.

As illustrated in FIG. 13A, there is a significant difference between the 1^{st} pre-test measurement value (i.e. the measurement between t₀ and t₁) and the 1^{st} post-test measurement value (i.e. the measurement after t₂). Therefore, the target material 400 is bind to the first probe 300a in the first sensor array SA1, and the first sensor array SA1 is marked as a binding sensor array. On the other hand, as illustrated in FIG. 13B to FIG. 13D, there is no significant different between the 2^{nd} pre-test measurement value (i.e. the measurement between t₁ and t₂ in FIG. 13B) and the 2^{nd} post-test measurement value (i.e. the measurement after t₂ in FIG. 13B), between the 3^{rd} pre-test measurement value (i.e. the measurement between t₁ and t₂ in FIG. 13C) and the 3^{rd} post-test measurement value (i.e. the measurement after t₂ in FIG. 13C), and between the 4^{th} pre-test measurement value (i.e. the measurement between t₁ and t₂ in FIG. 13D) and the 4^{th} post-test measurement value (i.e. the measurement after t₂ in FIG. 13D), so the target material 400 is not bind to the second probe 300b in the second sensor array SA2, the third probe 300c in the third sensor array SA3, and the fourth probe 300d in the fourth sensor array SA4. Please be noted that the slight differences between the 2^{nd} pre-test measurement value and the 2^{nd} post-test measurement value, between the 3^{rd} pre-test measurement value and the 3^{rd} post-test measurement value, and between the 4^{th} pre-test measurement value and the 4^{th} post-test measurement value are originated from noise or marginal error (derived from the washing step or other factors), and can be negligible. Since the first sensor array SA1 is being marked as the binding sensor array, the type of the target material 400 may be identified based on the type of the first probe 300a.

In some embodiments, by utilizing different sensor arrays (i.e. the first sensor array SA1, the second sensor array SA2, the third sensor array SA3, and the fourth sensor array SA4) with various assays (i.e. the first assay A1, the second assay A2, the third assay A3, and the fourth assay A4) at once, one time test may be performed. As such, the testing efficiency may be sufficiently enhanced. For example, the experimental time may be reduced to 15 minutes or less.

Please be noted that although the target material identification method shown in FIG. 12 and FIG. 13A to FIG. 13D utilizes four sensor arrays SA1-SA4 with four assays A1-A4, the disclosure is not limited thereto. Depending on the number of different probes, the number of the assays/the sensor arrays may vary. For example, the number of the assays/the sensor array may be ten, hundreds, thousands, or so as long as these assays/sensor arrays are all coated with different types of probes.

In accordance with some embodiments of the disclosure, a target material identification method includes at least the following steps. A bio-sensing integrated circuit having a sensor array is provided. The sensor array is divided into a 1^{st} assay to an N^{th} assay, and the 1^{st} assay to the N^{th} assay are coated with different probes. A calibration process is performed on the 1^{st} assay to the N^{th} assay to obtain a 1^{st} pre-test measurement value to an N^{th} pre-test measurement value respectively for the 1^{st} assay to the N^{th} assay. A sample fluid having the target material therein is applied onto the 1^{st} assay to the N^{th} assay. A bio-sensing process is performed on the sample fluid by the bio-sensing integrated circuit to obtain a 1^{st} post-test measurement value to an N^{th} post-test measurement value respectively for the 1^{st} assay to the N^{th} assay. The 1^{st} pre-test measurement value to the N^{th} pre-test measurement value are compared with the corresponding 1^{st} post-test measurement value to the corresponding N^{th} post-test measurement value, so as to determine whether the target material is bind to the probes in each of the 1^{st} assay to the N^{th} assay. An assay among the 1^{st} assay to the N^{th} assay having the target material bind to the probe is marked as a binding assay. The target material is identified based on the probe in the binding assay.

In accordance with some alternative embodiments of the disclosure, a target material identification method includes at least the following steps. A 1^{st} bio-sensing integrated circuit to an N^{th} bio-sensing integrated circuit are provided. The 1^{st} bio-sensing integrated circuit to the N^{th} bio-sensing integrated circuit are coated with different probes. A calibration process is performed on the 1^{st} bio-sensing integrated circuit to the N^{th} bio-sensing integrated circuit to obtain a 1^{st} pre-test measurement value to an N^{th} pre-test measurement value respectively for the 1^{st} bio-sensing integrated circuit to the N^{th} bio-sensing integrated circuit. A sample fluid having the target material therein is applied onto the 1^{st} bio-sensing integrated circuit to the N^{th} bio-sensing integrated circuit. A bio-sensing process is performed on the sample fluid by the 1^{st} bio-sensing integrated circuit to the N^{th} bio-sensing integrated circuit to obtain a 1^{st} post-test measurement value to an N^{th} post-test measurement value respectively for the 1^{st} bio-sensing integrated circuit to the N^{th} bio-sensing integrated circuit. The 1^{st} pre-test measurement value to the N^{th} pre-test measurement value are compared with the corresponding 1^{st} post-test measurement value to the corresponding N^{th} post-test measurement value, so as to determine whether the target material is bind to the probes in each of the 1^{st} bio-sensing integrated circuit to the N^{th} bio-sensing integrated circuit. The target material is identified based on the probe that is bind to the target material.

In accordance with some alternative embodiments of the disclosure, a target material identification method includes at least the following steps. A bio-sensing integrated circuit having a sensor array is provided. The sensor array includes a first assay having first pixels and a second assay having second pixels. A coating process is performed to coat a first probe onto the first pixels and to coat a second probe onto the second pixels. The first probe is different from the second probe. A first calibration process is performed on the first assay to obtain a 1^{st} pre-test measurement value. A second calibration process is performed on the second assay to obtain a 2^{nd} pre-test measurement value. A sample fluid having the target material therein is applied onto the first assay and the second assay. A first bio-sensing process is performed on the sample fluid in the first assay by the bio-sensing integrated circuit to obtain a 1^{st} post-test measurement value. A second bio-sensing process is performed on the sample fluid in the second assay by the bio-sensing integrated circuit to obtain a 2^{nd} post-test measurement value. The 1^{st} pre-test measurement value is compared with the 1^{st} post-test measurement value to determine whether the target material is bind to the first probe in the first assay. The 2^{nd} pre-test measurement value is compared with the 2^{nd} post-test measurement value to determine whether the target material is bind to the second probe in the second assay. The target material is identified based on the first probe or the second probe that is bind to the target material.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A target material identification method, comprising:
providing a bio-sensing integrated circuit having a sensor array, wherein the sensor array is divided into a 1^{st} assay to an N^{th} assay, and the 1^{st} assay to the N^{th} assay are coated with different probes;
performing a calibration process on the 1^{st} assay to the N^{th} assay to obtain a 1^{st} pre-test measurement value to an N^{th} pre-test measurement value respectively for the 1^{st} assay to the N^{th} assay;
applying a sample fluid having the target material therein onto the 1^{st} assay to the N^{th} assay;
performing a bio-sensing process on the sample fluid by the bio-sensing integrated circuit to obtain a 1^{st} post-test measurement value to an N^{th} post-test measurement value respectively for the 1^{st} assay to the N^{th} assay;
comparing the 1^{st} pre-test measurement value to the N^{th} pre-test measurement value with the corresponding 1^{st} post-test measurement value to the corresponding N^{th} post-test measurement value, so as to determine whether the target material is bind to the probes in each of the 1^{st} assay to the N^{th} assay;
marking an assay among the 1^{st} assay to the N^{th} assay having the target material bind to the probe as a binding assay; and
identifying the target material based on the probe in the binding assay.

2. The method of claim 1, wherein the bio-sensing integrated circuit comprises Biosensor Field-Effect Transistors (BioFETs), the sensory array comprises pixels arranged in an array, and each BioFET corresponds to a pixel.

3. The method of claim 2, wherein the probes are coated in each of the pixels.

4. The method of claim 2, wherein each BioFET comprises a drain region and a source region, and the 1^{st} pre-test measurement value to the N^{th} pre-test measurement value and the 1^{st} post-test measurement value to the N^{th} post-test measurement value are currents between the source region and the drain region.

5. The method of claim 4, wherein the 1^{st} pre-test measurement value is an average value of the currents between the source region and the drain region of each pixel in the 1^{st} assay of the sensor array.

6. The method of claim 4, wherein the 1^{st} post-test measurement value is an average value of the currents between the source region and the drain region of each pixel in the 1^{st} assay of the sensor array.

7. The method of claim 1, wherein the 1^{st} pre-test measurement value to the N^{th} pre-test measurement value are obtained in sequential order.

8. The method of claim 1, wherein the 1^{st} pre-test measurement value to the N^{th} pre-test measurement value are obtained simultaneously.

9. A target material identification method, comprising:
providing a 1^{st} bio-sensing integrated circuit to an N^{th} bio-sensing integrated circuit, wherein the 1^{st} bio-sensing integrated circuit to the N^{th} bio-sensing integrated circuit are coated with different probes;
performing a calibration process on the 1^{st} bio-sensing integrated circuit to the N^{th} bio-sensing integrated circuit to obtain a 1^{st} pre-test measurement value to an N^{th} pre-test measurement value respectively for the 1^{st} bio-sensing integrated circuit to the N^{th} bio-sensing integrated circuit;
applying a sample fluid having the target material therein onto the 1^{st} bio-sensing integrated circuit to the N^{th} bio-sensing integrated circuit;
performing a bio-sensing process on the sample fluid by the 1^{st} bio-sensing integrated circuit to the N^{th} bio-sensing integrated circuit to obtain a 1^{st} post-test measurement value to an N^{th} post-test measurement value respectively for the 1^{st} bio-sensing integrated circuit to the N^{th} bio-sensing integrated circuit;
comparing the 1^{st} pre-test measurement value to the N^{th} pre-test measurement value with the corresponding 1^{st} post-test measurement value to the corresponding N^{th} post-test measurement value, so as to determine whether the target material is bind to the probes in each of the 1^{st} bio-sensing integrated circuit to the N^{th} bio-sensing integrated circuit; and
identifying the target material based on the probe that is bind to the target material.

10. The method of claim 9, wherein each of the 1^{st} bio-sensing integrated circuit to the N^{th} bio-sensing integrated circuit respectively comprises Biosensor Field-Effect Transistors (BioFETs), each BioFET comprises a drain region and a source region, and the 1^{st} pre-test measurement value to the N^{th} pre-test measurement value and the 1^{st} post-test measurement value to the N^{th} post-test measurement value are currents between the source region and the drain region.

11. The method of claim 10, wherein the 1^{st} pre-test measurement value is an average value of the currents between the source region and the drain region of each bioFET in the 1^{st} bio-sensing integrated circuit.

12. The method of claim 10, wherein the 1^{st} post-test measurement value is an average value of the currents between the source region and the drain region of each bioFET in the 1^{st} bio-sensing integrated circuit.

13. The method of claim 9, wherein the 1^{st} bio-sensing integrated circuit to the N^{th} bio-sensing integrated circuit are placed on a same cartridge.

14. The method of claim 9, wherein the 1^{st} pre-test measurement value to the N^{th} pre-test measurement value are obtained in sequential order.

15. The method of claim 9, wherein the 1^{st} pre-test measurement value to the N^{th} pre-test measurement value are obtained simultaneously.

16. A target material identification method, comprising:
providing a bio-sensing integrated circuit having a sensor array, wherein the sensor array comprises a first assay having first pixels and a second assay having second pixels;
performing a coating process to coat a first probe onto the first pixels and to coat a second probe onto the second pixels, wherein the first probe is different from the second probe;
performing a first calibration process on the first assay to obtain a 1^{st} pre-test measurement value;
performing a second calibration process on the second assay to obtain a 2^{nd} pre-test measurement value;
applying a sample fluid having the target material therein onto the first assay and the second assay;
performing a first bio-sensing process on the sample fluid in the first assay by the bio-sensing integrated circuit to obtain a 1^{st} post-test measurement value;
performing a second bio-sensing process on the sample fluid in the second assay by the bio-sensing integrated circuit to obtain a 2^{nd} post-test measurement value;
comparing the 1^{st} pre-test measurement value with the 1^{st} post-test measurement value to determine whether the target material is bind to the first probe in the first assay;
comparing the 2^{nd} pre-test measurement value with the 2^{nd} post-test measurement value to determine whether the target material is bind to the second probe in the second assay; and
identifying the target material based on the first probe or the second probe that is bind to the target material.

17. The method of claim 16, wherein the coating process comprises:
performing a surface activation process on the sensor array of the bio-sensing integrated circuit;
placing a first mask layer on the sensor array, wherein the first mask layer comprises first openings exposing the first pixels of the first assay;
coating a first probe onto the first pixels exposed by the first openings;
removing the first mask layer;
placing a second mask layer on the sensor array, wherein the second mask layer comprises second openings exposing the second pixels of the second assay;
coating a second probe onto the second pixels exposed by the second openings; and
removing the second mask layer.

18. The method of claim 17, wherein the surface activation process is performed globally on the first assay and the second assay simultaneously.

19. The method of claim 16, wherein the first calibration process is performed before the second calibration process.

20. The method of claim 16, wherein the first calibration process and the second calibration process are performed simultaneously.
